(19) United States
(12) Patent Application Publication
(10) Pub. No.: US 2003/0003097 A1
(43) Pub. Date: Jan. 2, 2003

(54) Recombinant antibodies coexpressed with GnTIII

(75) Inventors: Mitchell E. Reff, San Diego, CA (US); Julian Davis, San Diego, CA (US)

Correspondence Address: PILLSBURY WINTHROP, LLP, P.O. BOX 10500, MCLEAN, VA, 22102, US

(73) Assignee: IDEC Pharmaceutical Corporation

(21) Appl. No.: 10/113,929

(22) Filed: Apr. 2, 2002

Related U.S. Application Data
(60) Provisional application No. 60/280,139, filed on Apr. 2, 2001.

(51) Int. Cl. A61K 39/395; C12N 5/06; C07K 16/30

(52) U.S. Cl. 424/144.1; 435/334; 530/388.22

(57) ABSTRACT

Methods, compostions and kits comprising antibodies for the treatment of neoplastic, autoimmune or other disorders are provided.

### CROSS REFERENCE TO RELATED APPLICATIONS

This application claims priority to U.S. Provisional Application No. 60/280,139 filed Apr. 2, 2001, which is incorporated herein by reference in its entirety.

### FIELD OF THE INVENTION

In a broad aspect the present invention generally relates to antibodies produced by eukaryotic cell lines that express GnTIII and a recombinant antibody.

### BACKGROUND OF THE INVENTION

Improved methodologies for maximizing recombinant gene expression is an on-going effort in the art. Of particular interest is the development of methodologies that maximize recombinant expression of mammalian genes suitable for producing commercially useful quantities of biologically active proteins. While prokaryotic, typically bacterial, host cell systems have proven capable of generating large quantities of recombinant proteins, these hosts suffer from a number of disadvantages, including an inability to glycosylate proteins, inefficient cleavage of “pre” or “prepro” sequences from proteins (e.g., inefficient post translational modification), and a general inability to secrete proteins. Consequently the art has sought eukaryotic host systems, typically mammalian host cell systems, for mammalian protein production. One feature of such systems is that the protein produced has a structure most like that of the natural protein species, and, purification often is easier since the protein can be secreted into the culture medium in a biologically active form.

A number of problems still exist however, in mammalian culture systems. Specifically, high levels of expression typically are not easily obtained in mammalian systems. In addition, eukaryotic host cells typically have more stringent requirements for culturing and have slower growth rates. Thus, producing large quantities of a recombinant protein requires more than simply culturing a host cell transfected with an expression vector. This is particularly true when the gene of interest is a poorly expressed gene, i.e., is not produced in abundance or is only transiently expressed under natural, physiological conditions. The genes encoding these proteins typically have multiple levels of regulation, often at one or more levels of the expression system, e.g., at the level of transcription, translation, post translation modification, secretion and/or activation. Typically these genes, when stably integrated in unamplified, immortalized cells, produce less than about 10-100 ng protein/10⁶ cells per ml. Maximizing production of these proteins means identifying means for circumventing these levels of regulation.

Through protein engineering it has become possible to fashion antibodies in a variety of ways. Changes in immunogenicity, affinity, valency and effector funtion have all been achieved by standard molecular biology techniques (for review see (Gavilondo and Larrick 2000; Hollinger and Bohlen 1999)). Furthermore, such techniques have aided the approval of antibodies for therapeutic use (Hollinger and Bohlen 1999; Newman and Ryskamp 1999). Rituxan® is an example of such an antibody, a chimeric mouse human IgGI-κ antibody approved for use in relapsed or refractory, low grade follicular B cell non-Hodgkins lymphoma (Maloney et al. 1997; Reff et al. 1994). The antibody recognizes CD20, a 35 kD cell surface phosphoprotein (Valentine et al. 1989) expressed on neoplastic B Cells and has been shown to mediatem complement dependant cytotoxicity, (CDC) antibody dependant cellular cytotoxicity (ADCC) in-vitro (Reff et. al. 1994) and induce apoptosis of tumor cell lines when crosslinked (Maloney et. al. 1997; Shan et al. 1998).

There remains a need for enhanced methods of production, expression and stabilization of antibodies.

### SUMMARY OF THE INVENTION

In one embodiment the invention provides an eukaryotic cell line that expresses GnTIII and a recombinant antibody, preferably, the eukaryotic cell line is a mammalian cell line, most preferably a CHO cell line. The antibody is a human, chimeric or humanized antibody, preferably an anti-CD20 antibody. A particularly preferred antibody is RITUXAN®.

In a particularly preferred embodiment, the antibody reacts with a tumor associated antigen. More particularly, a tumor associated antigen selected from the group consisting of CD2, CD3, CD5, CD6, CD7, MAGE-1, MAGE-3, MUC-1, HPV 16, HPV E6, HPV E7, TAG-72, CEA, L6-Antigen, CD19, CD20, CD22, CD37, CD52, HLA-DR, EGF receptor and HER2 Receptor.

In another embodiment the invention provides an antibody produced by a cell line eukaryotic cell line that expresses GnTIII and a recombinant antibody.

In yet another embodiment, the invention provides therapies including the administration of an antibody produced by provides an eukaryotic cell line that expresses GnTIII and a recombinant antibody. Particularly, the invention provides therapies of a neoplastic disorder such as a disorder selected from the group consisting of relapsed Hodgkin's disease, resistant Hodgkin's disease high grade, low grade and intermediate grade non-Hodgkin's lymphomas, B cell chronic lymphocytic leukemia (B-CLL), lymphoplasmacytoid lymphoma (LPL), mantle cell lymphoma (MCL), follicular lymphoma (FL), diffuse large cell lymphoma (DLCL), Burkitt's lymphoma (BL), AIDS-related lymphomas, monocytic B cell lymphoma, angioimmunoblastic lymphoadenopathy, small lymphocytic; follicular, diffuse large cell; diffuse small cleaved cell; large cell immunoblastic lymphoblastoma; small, non-cleaved; Burkift's and non-Burkitt's; follicular, predominantly large cell; follicular, predominantly small cleaved cell; and follicular, mixed small cleaved and large cell lymphomas. Therapies according to the invention also include treating an immune disorder.

In yet another embodiment, the invention also provides a kit useful for the treatment of a mammal suffering from or predisposed to a disorder comprising at least one container having a antibody produced by provides an eukaryotic cell line that expresses GnTIII and a recombinant antibody deposited therein and a label or an insert indicating that said antibody may be used to treat said disorder.

In yet a further embodiment, the invention provides a method for forming antibodies comprising the steps of:

culturing prokaryotic or eukaryotic host cells comprising DNA sequences encoding GnTIII and a recombinant antibody antibody whereby the host cell expresses GnTIII and the recombinant antibody;

allowing the host cell to express GnTIII and the recombinant antibody; and

recovering said antibodies from the host cell culture.

In yet another embodiment, the invention provides a polycistronic vector for expressing GnTIII and functional antibodies in eukaryotic host cells which vector comprises a polycistronic transcription system comprising a DNA sequence encoding GnTIII and the following elements operably linked in the 5′ to 3′ orientation:

(i) a promoter operable in a eukaryotic cell;

(ii) a DNA sequence encoding an antibody light chain which optimally comprises at its 5′ terminus a signal peptide coding sequence operable in eukaryotic cells which DNA sequence does not comprise at its 3′ end a poly A sequence and comprising a start and a stop codon at the 5′ and 3′ terminus of said DNA sequence;

(iii) an internal ribosome entry site (IRES) obtained from a member selected from the group consisting of a cardiovirus, a herpes virus and a poliovirus; and

(iv) at least one DNA sequence comprising the following elements (a) a DNA sequence encoding an antibody heavy chain wherein said DNA optimally comprises at its 5′ terminus a signal peptide coding sequence operable in eukaryotic cells and wherein said DNA sequence comprises a poly A sequence at its 3′ terminus only if the DNA sequence is the 3′ most coding sequence in the polycistron, and further comprises a start and stop codon at the 5′ and 3′ termini of said DNA coding sequence;

wherein the DNA sequence encoding the antibody light chain is expressed at a ratio ranging between 10:1 and 1:1 with respect to the DNA sequence encoding the antibody heavy chain.

### BRIEF DESCRIPTION OF THE FIGURES

FIG. 1. Schematic representation of the vector constructed to allow constitutive expression of rat GnTIII in mammalian cells. CMV, cytomegalovirus promoter; BGH, Bovine growth hormone poly adenylation; SV, SV40 early polyadenylation; SVO, SV40 Ori origin, GnIII rat GnTIII gene; Pur R Puromycin resistance gene; Beta Lac, Beta lactamase gene; F1 Ori, F1 origin of replication; Col E1 Ori, ColEl compatibility group origin of replication.

FIG. 2. Detection of GnTIII mRNA by RT Relative QPCR Lower bands are that of the 18S internal standard. Upper band is that of a 500 by fragment of GnTIII. Using the intensity of the 18S as a reference some clones were scored as having high mRNA for GnTIII (eg 50C9-1A12) and one clone (50C9-1A7) having low levels of GnTIII mRNA. 6B4 is an immunoglobulin expressing cell line inducible for GnTIII which was previously isolated in our laboratory and used here as a positive control. 50C9 is the parent untransfected cell line. C=negative control with no cDNA template included in the PCR.

FIG. 3 HPLC analysis of oligosaccharides isolated from antibody preparations from A) 50C9 and B) 50C9-1B9. Inserts show structures associated with the peaks. GN*=Bisecting G1cNAc, GN=G1cNac, G=Galctose, M=Mannose, F=Fucose.

FIG. 4 Comparison of preparation with Bisected glycoforms mAbs 50C9-1A12, 50C9-1A7, 50C9-1B9 mAbs and the parent mAb 50C9 in ADCC against B cell Antigen CD20. 50C9-1A12, 50C9-1A7, 50C9-1B9 mAbs and 50C9 mAb were compared in their capacity to mediate ADCC against SKW 6.4 target cells at an E:T of 80:1. The effector cells PBMC from healthy donors were treated with 10 u/ml of IL-2 for overnight. As figure shown effector cells populations with irrelevant mAb were not able to kill target cells. 50C9 anti-CD20 mAb and Bisected glycoforms preparation mAbs 50C9-1A12, 50C91A7, 50C9-IB9 was significant mediated effector cells to kill target cells. The Bisected glycoforms altered mAbs events have higher capacity to mediate ADCC against SKW6.4 cell than the parent mAb 50C9 The half max effective function are 10-20 fold higher. The results are presented as mean ±sem (indicated by error bars

FIG. 5 Blocking FcγR receptors on PBMC in 50C9-IA7 mAb mediated ADCC assays. Effector cells (PBMC) were pre-incubated with either Anti-FcγRIII mAb or anti-FcγRI mAb. Anti-FcγRIII mAb abolished 50C9-1A7 mediated ADCC activity. PBMC pretreated with anti-FcγRI mAb has no effect on 50C9-1A7 mAb mediated ADCC .

FIG. 6 Binding of 50C9 and 50C9-1A7 on FcγRIII positive NK cells. FcγRIII positive NK cells were covalently attached on the 96 a flat bottom plate and fixed with 0.5% glutaraldehyde. 50C9 and 50C9-IA7 diluted in blocking buffer were added to the plate in triplicate wells and incubated at 37° C. for one hour. After several washes, the binding of was detected by using anti-human IgG Fab fragments conjugated with horse radish peroxidase and developed with tetramethylbenzidine. The results are presented as mean ±sem.

### DETAILED DESCRIPTION OF THE INVENTION

While the present invention may be embodied in many different forms, disclosed herein are specific illustrative embodiments thereof that exemplify the principles of the invention. It should be emphasized that the present invention is not limited to the specific embodiments illustrated.

The present invention is predicated, at least in part, on the inventors' unexpected discovery that it is possible to isolate useful highly productive recombinant cell lines which co-express a glycotransferase. Furthermore, the inventors have discovered that the catalytic activity of the enzyme is high enough to effect the biological activity of the co-expressed recombinant protein without greatly effecting growth or expression levels. More specifically it has surprisingly been found that the increase in ADCC described herein allows the use of lower doses of immunoglobulin but with similar therapeutic effects as the wild type parent.

The advantages of the invention are exemplified below in conjunction with B cell tumors which express lower levels of CD20 and which require high doses of the current antibody to be effective. The synergistic effects afforded by the present invention allow the use of the anti CD20 antibody at significantly lower levels thereby reducing toxicity related side effects associated with high dosage therapeutic regimens.

Recent studies have shown that engineering the glycoforms of immunoglobulins may also yield optimized effector functions. The N-acetyl glucosaminyl transferase III (GnTIII) enzyme has been expressed in a Chinese hamster ovary (CHO) cell line expressing an anti-neuroblastoma IgGI resulting in greater antibody dependant cellular cytotoxic (ADCC) activity (Umana et al. 1999a). GnTIII is a golgi localized enzyme and catalyses the addition of a N-actetylglucosamine (G1cNAC) residue to a bisecting position of N linked oligosaccharide chains (Narisimhan 1982). This particular modification is commonly found in the N-linked sugar residues of human IgG but not of other mammalian species and the bisecting sugar has been implicated in biological activity of therapeutic antibodies (Lifely et al. 1995). However, published studies have concentrated on cell lines producing very small amounts of antibody and the co-expression of the enzyme in cell lines producing large amounts of antibody (production cell lines) has not been reported. Furthermore, recent reports have indicated that due to growth inhibition effects regulated enzyme expression may be required for successful expression in production cell lines (Umana et al. 1999b)

As discussed in more detail below, the present inventors describe herein the first use of GnTIII over-expression in a production cell line. Rituxan® is currently produced at high levels in CHO cells in which endogenous hamster GnTIII is not expressed. The wild type antibodies therefore, contain biantennary oligosacchraide with no bisecting G1cNAc residues. Following co-expression of GnTIII in the production cell line the effect on cell growth kinetics, antibody production levels, glycoform composition and functional changes in ADCC activity were measured.

Recent efforts to co-express GnTIII in recombinant mammalian cells have concentrated on cell lines which express only small amounts of recombinant protein (Bailey et al. 1997; Umana et. al. 1999a)). A recent report has also indicated that the over-expression of glycotransferases in mammalian cells leads to an inhibition of cell growth ((Umana et. al. 1999b)). Thus, the present inventors hypothesized that regulated gene expression may be required to achieve efficient expression in production cell lines. As discussed below, cell lines have been constructed in which GnTIII was controlled in an inducible fashion. The inventors unexpectedly found that the low level of basel expression in such cell lines had enough effect on antibody glycosylation without great effects on cell growth to warrant the use of constitutive expression systems.

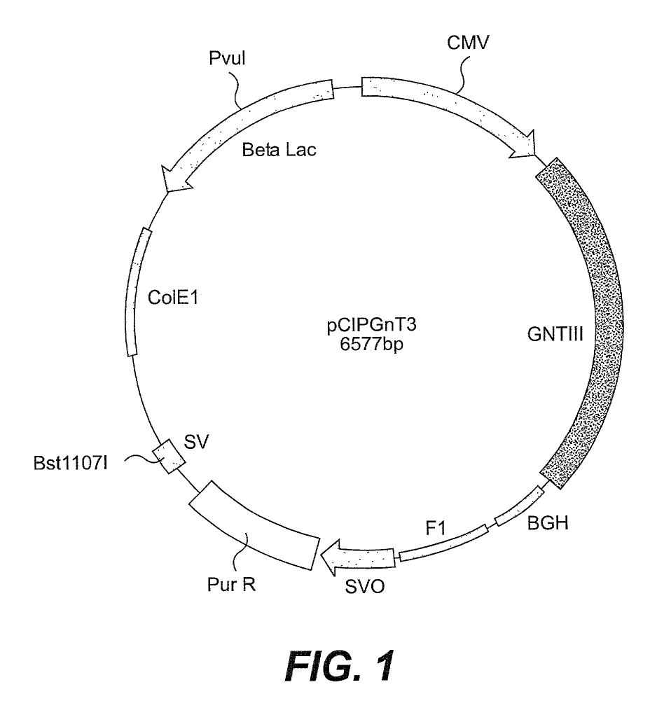

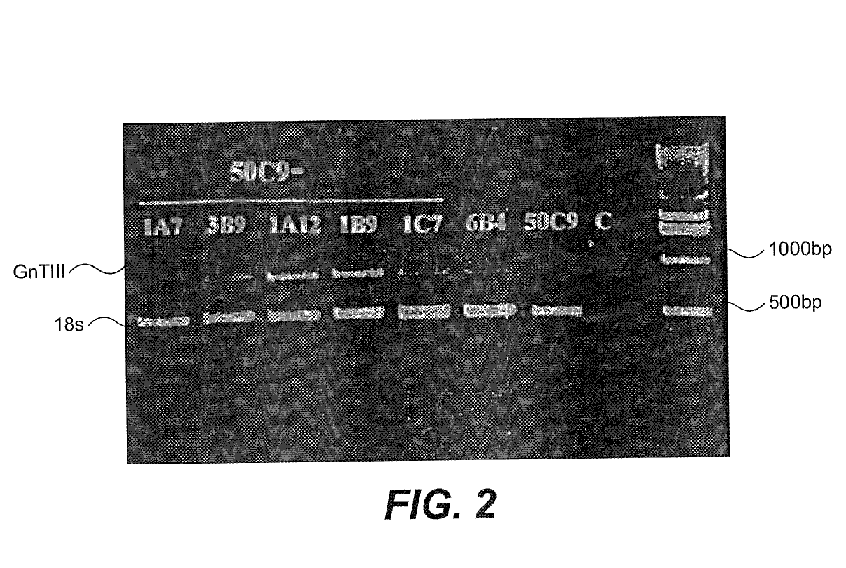

As discussed in connection with Example 1, the inventors constructed a constitutive expression plasmid for GnTIII. The plasmid pCIPGnT3 (FIG. 1) contains the rat GnTIII gene under a constitutive CMV promoter and bovine growth hormone polyadenylation region. The plasmid also contains a puromycin resitance gene, which allows selection of in puromycin containing media. Following electroporation of the Rituxan® producing cell line (50C9) with the plasmid, puromycin resistant colonies were obtained. We then employed a relative QPCR assay to detect message RNA levels in the resistant colonies to decide which clones to study further. Most of the clones isolated expressed high levels of GnTIII message and a few clones expressed at much lower levels. An example of a relative QPCR experiment is shown in FIG. 2. Clone 50C9-IA7 is an example of a clone which expresses at a lower level whilst 50C9-1A12 and 50C9-1B9 are clones which express at much higher levels. No GnTIII message was detected for the parent cell line (not transfected with the pCIPGnT3 plasmid) indicating the absence of endogenous GnTIII expression in this cell line.

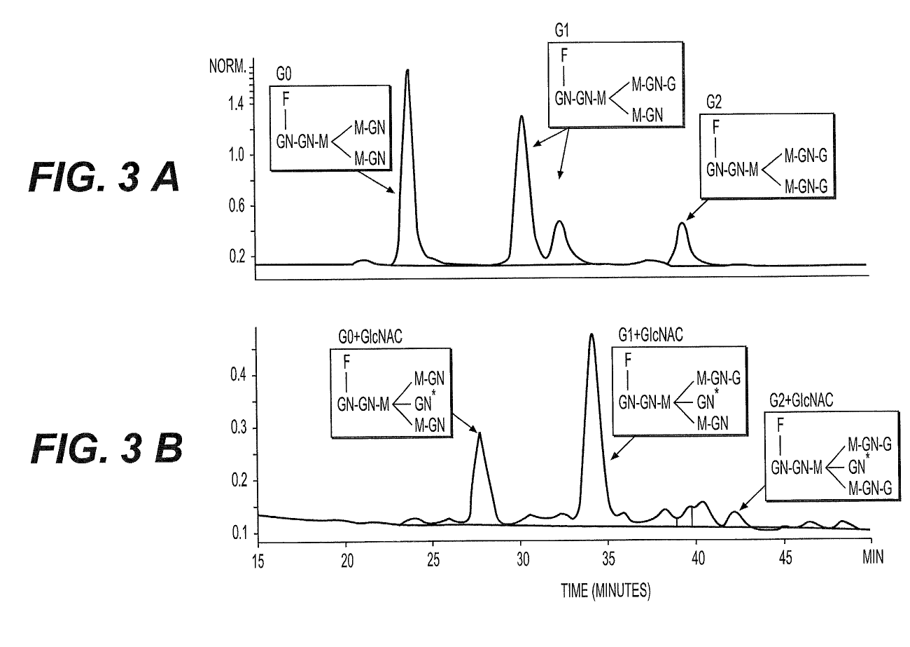

Three clones were then chosen to study the in-vivo catalytic effects of GnTIII on the glycoforms of purified antibody by HPLC analysis. All three showed considerable glycoform variation when compared to the parent (50C9) cell line. A typical HPLC trace for the parent and one GnTIII transfectant clone is shown in FIG. 3. No bisected glycoforms were found for the 50C9 cell line. However, for the GnTIII positive cell line the majority of glycoforms (48-71%) contain a bisected GlcNac residue. A full set of results for the three cell lines are shown in Table 1. The data shows that there are small differences in the glycoform composition of GnTIII transfected clones but that for all three the dominant glycoform species found was a bisected biantennary oligosaccharide with one galactose residue (G1+G1cNAc). Only small amounts (3-5%) of bisected biantennary oligosaccharide with two galactose residues were detected (G2+G1cNAc).

As indicated above, previous reports have suggested that the over-expression of glycotransferase in mammalian cells leads to slower growth kinetics setting an upper limit on the amount of glycosyltransferase which may be expressed in a cell line (Umana et. al. 1999b). We therefore studied the growth kinetics and production levels in the three GnTIII positive cell lines and compared them with 50C9 which produces large amounts of immunolglobulin (pcd) and has very good growth kinetics (td). We found all three cell lines to have very good expression levels of immunolglobulin. Furthermore, all three cell lines also have favorable growth kinetics and one clone (50C9-1A12) has growth kinetics (i.e. doubling time) very similar to the parent antibody (Table II). The summary of data shown in Table II indicates no correlation between mRNA levels and GnTIII activity or doubling time in the cell lines.

The high mRNA levels and GnTIII activity found in clone 50C9-1A12 do not seem to effect the growth kinetics or antibody expression levels. These results differ from those published previously in which the level of GnTIII expression correlated to growth inhibition. (Umana et. al. 1999b). Without wishing to be bound to any explanation or theory, the present inventors offer two possible reasons for the growth inhibition effect. Either a direct effect of protein over-expression leading to inhibition which is independent of the catalytic activity of GnTIII or a direct effect of the in-vivo catalytic activity of GnTIII on endogenous proteins. The previous work in this field has used glycotransferase co-expression in cell lines expressing only small amounts of recombinant protein. The work presented here uses a production cell line which produces large amount of immunoglobulin and supports the latter suggestion for growth inhibition. The former suggestion seems unlikely since production cell lines should be more sensitive to growth inhibition due to the over-expression of an additional protein. The absence of growth inhibition observed here may be linked to the cells high production of immunoglobulin which may occupy the over-expressed GnTIII and prevent its catalytic activity on other endogenous proteins.

The anti-CD20 antibody used in Example 1 is approved as a therapeutic agent in non-Hodgkin's lymphoma and has been shown to produce effective responses in approximately 50% of patients through depletion of normal and malignant B cells. The possible mechanisms include complement dependant cytotoxicity (CDC), antibody dependant cellular cytotoxicity (ADCC) and induction of apoptosis of CD20 positive cells on binding of the antibody. The over-expression of GnTIII has led to the isolation of clones with bisecting glycoform hybrids which are absent in the parent. These bisecting glycoforms have been implicated in the biological activity of some antibodies and so the antibodies purified from the 50C9/GnTIII clones were studied for changes in biological activity. No differences due to complement binding or apoptosis of CD20 positive cells on antibody binding were observed for the glycoform altered antibodies (data not shown). However, antibodies produced by all three GnTIII transfected cell lines studied were as effective as antibodies produced by the wild type cell line in killing CD20 positive target cells but at a 10 to 20 times lower concentration. This agrees with results reported for the over-expression of GnTIII in a cell line expressing an anti-neuroblastoma IgG.(Umana et. al. 1999a) In the aforementioned report the glycoforms of an antibody with low ADCC activity were altered resulting in higher ADCC activity and making it more attractive for therapeutic use (Umana et. al. 1999a).

Here we have taken an approved therapeutic antibody with good ADCC activity and improved it further. This may allow the use of the antibody at lower doses with no reduction in efficacy. Moreover, the higher ADCC activity at a lower antibody concentration may result in an enhanced response in lymphomas and leukemias expressing lower levels of the CD20 antigen. Certain forms of these diseases require high doses of the current drug to be effective. It is expected that other antibodies deficient in the bisecting glycoforms may improve their in vivo cytolytic function.

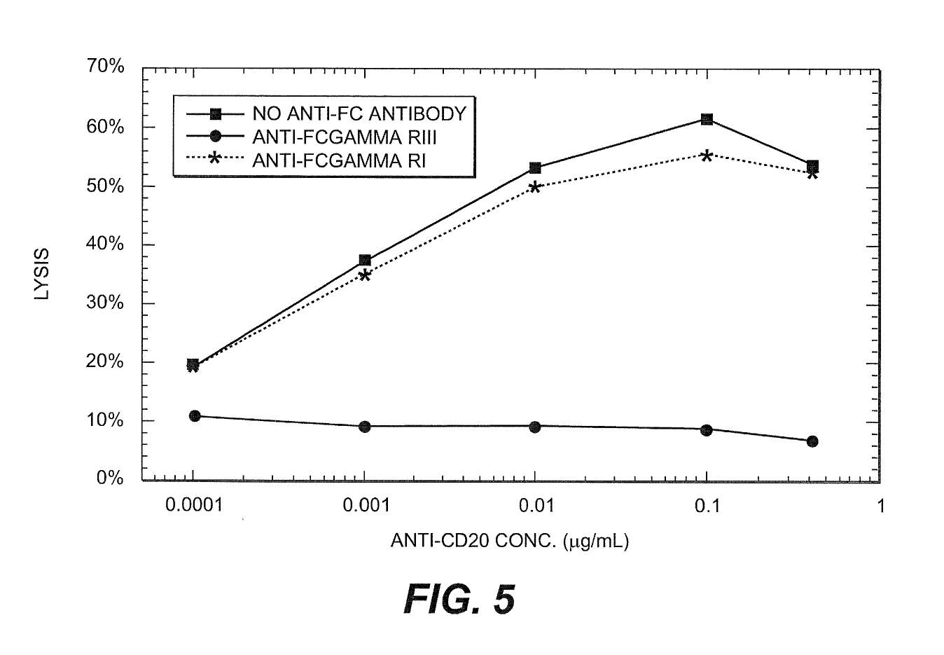

The ADCC activity observed for the anti-CD20 antibody is believed to be a result of specific killing of antigen positive cells by NK cells through binding of the IgG1Fc domain to FcγRIII receptors. We therefore used an anti-FcγRIII antibody (reported to block Fc binding) to specifically block the FcγRIII receptors on NK cells and study the effect on ADCC activity. Blocking NK cells with the anti-FcγRIII antibody abolished the ADCC activity of both 50C9 and 50C9-1A7. FIG. 5 shows the results obtained from the 50C9-1A7 antibody preparation. No inhibition of ADCC was observed in an experiment in which PBMC cells were pre-incubated with an antibody against FcγRI which is reported to block Fc binding to the FcγRI receptor. The data suggests therefore that FcγRI receptors are not involved in the ADCC activity of the 50C9-IA7 antibody preparation with bisected biantennary oligosaccarides.

Without wishing to be bound to any explanation or theory, two possible explanations why antibodies with bisected biantennary oligosaccarides give rise to better ADCC activity than those in which the glycoforms are absent are suggested by the results provided herein. Firstly, the effect may be due to a simple increase in affinity of the altered antibody for the FcyRIII receptor. Secondly, better ADCC may also result from a better crosslinking of FcγRIII receptors on the surface of NK cells which initiates the process of degranulation leading to lysis of the target cell.

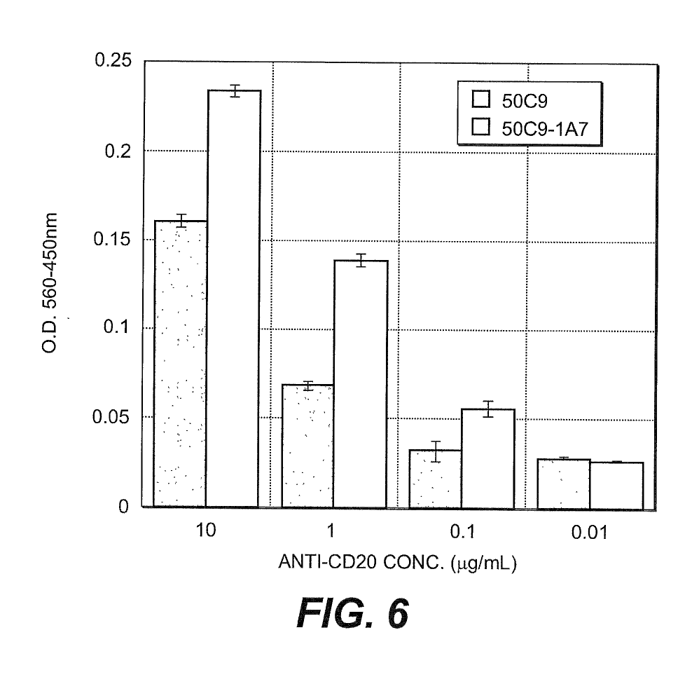

Using a whole cell ELISA, the binding characteristics of the parent and the GnTIII positive clone 50C9-1A7 were evaluated. Antibodies from 50C9-1A7 bound better to NK cells than antibodies prepared from the parent 50C9 (FIG. 6). The antibody also bound better to FcγRI and FcγRII expressing cells (results not shown). However, the role of FcγRI in ADCC has been discounted in the previously described experiment (FIG. 5). The increase in ADCC activity is therefore most likely due to increased binding of the antibody to FcyRIII on NK cells. Since no CD20 antigen was present in the ELISA, increased binding due to better crosslinking of IgG can be ruled out. Presumably the increase in binding is due to conformational effects specified by the bisecting glycoform on the Fc structure of the antibody. Since the parent antibody already has good ADCC activity it probably has a near optimal confirmation for FcyRIII binding which is then ‘fine tuned’ by the addition of the bisecting G1cNAc in the N-linked bianntennary oligosaccharide structure.

As discussed in more detail below the term “modified antibody” shall be held to mean any antibody, or binding fragment or recombinant thereof, immunoreactive with a tumor associated antigen in which at least a fraction of one or more of the constant region domains has been deleted or otherwise altered so as to provide desired biochemical characteristics such as non-covalent association with similar molecules, increased tumor localization or reduced serum half-life when compared with a whole, unaltered antibody of approximately the same binding specificity. In preferred embodiments, the modified antibodies of the present invention have at least a portion of one of the constant domains deleted. Such constructs shall be termed “domain deleted antibodies” for the purposes of the instant disclosure. More preferably, one entire domain of the constant region of the modified antibody will be deleted and even more preferably the entire C_{H}2 domain will be deleted. As discussed herein, each of the desired variants may readily be fabricated or constructed from a whole precursor or parent antibody using well known techniques.

The disclosed genetically engineered antibodies of the invention due to their increased binding affinity over conventional constructs, would be useful in therapeutic applications which do not require cell depletion or killing but require blocking of the target antigen such as to prevent a ligand/receptor interaction. Exemplary uses would be blocking of CD4 cells using an anti-CD4 antibody or B7 interactions either by blocking B7 or its T-cell receptor, CTLA-4 or CD80. Other examples could include blocking the CD23 antigen using a version of IDEC 152 (an anti-CD23 antibody) or blocking the CD40-CD40L interaction using a version of IDEC 131 (an anti-CD40L antibody); and

The constructs would also have therapeutic application in viral or bacterial neutralization, given their high binding affinity over monomeric antibodies and their rapid accumulation and digestion in the liver. Many example can be considered including anti-RSV antibodies, anti-HPV antibodies and anti-HIV antibodies.

In addition to the uses enumerated above, those skilled in the art will appreciate that the compounds, compositions and methods of the present invention are particularly useful for treating a variety of disorders including neoplastic disorders or immune (including autoimmune) disorders. In this regard the present invention may be used to treat any neoplastic disorder, tumor or malignancy that exhibits a tumor associated antigen. Similarly, the methods and compositions may be used to treat any autoimmune disorder or anomaly caused in whole or in part by a cell population exhibiting an autoantigen.

As discussed above, the antibodies of the present invention may be immunoreactive with a tumor antigen or an antigen associated with immune disorders. For neoplastic disorders, the antigen binding portion (i.e. the variable region or immunoreactive fragment or recombinant thereof) of the disclosed antibodies binds to a selected tumor associated antigen at the site of the malignancy. Similarly, in immune (including autoimmune) disorders the disclosed antibodies will bind to selected markers on the offending cells. Given the number of reported antigens associated with neoplasms and immune disorders, and the number of related antibodies, those skilled in the art will appreciate that the presently disclosed antibodies may therefore be derived from any one of a number of whole antibodies. More generally, antibodies useful in the present invention may be obtained or derived from any antibody (including those previously reported in the literature) that reacts with an antigen or marker associated with the selected condition. Further, the parent or precursor antibody, or fragment thereof, used to generate the disclosed antibodies may be murine, human, chimeric, humanized, non-human primate or primatized. In other preferred embodiments the antibodies of the present invention may comprise single chain antibody constructs (such as that disclosed in U.S. Pat. No. 5,892,019 which is incorporated herein by reference) having altered constant domains as described herein. Consequently, any of these types of antibodies modified in accordance with the teachings herein is compatible with the instant invention.

As used herein, “tumor associated antigens” means any antigen which is generally associated with tumor cells, i.e., occurring at the same or to a greater extent as compared with normal cells. More generally, tumor associated antigens comprise any antigen that provides for the localization of immunoreactive antibodies at a neoplastic cell irrespective of its expression on non-malignant cells. Such antigens may be relatively tumor specific and limited in their expression to the surface of malignant cells. Alternatively, such antigens may be found on both malignant and non-malignant cells. For example, CD20 is a pan B antigen that is found on the surface of both malignant and non-malignant B cells that has proved to be an extremely effective target for immunotherapeutic antibodies for the treatment of non-Hodgkin's lymphoma. In this respect, pan T cell antigens such as CD2, CD3, CD5, CD6 and CD7 also comprise tumor associated antigens within the meaning of the present invention. Still other exemplary tumor associated antigens comprise but not limited to MAGE-1, MAGE-3, MUC-1, HPV 16, HPV E6 & E7, TAG-72, CEA, L6-Antigen, CD19, CD22, CD37, CD52, HLA-DR, EGF Receptor and HER2 Receptor. In many cases immunorecative antibodies for each of these antigens have been reported in the literature. Those skilled in the art will appreciate that each of these antibodies may serve as a precursor for modified antibodies in accordance with the present invention.

The antibodies of the present invention preferably associate with, and bind to, tumor or immune associated antigens as described above. Accordingly, as will be discussed in some detail below the antibodies of the present invention may be derived, generated or fabricated from any one of a number of antibodies that react with tumor associated antigens. In preferred embodiments the antibodies are modified or domain deleted antibodies that are derived using common genetic engineering techniques whereby at least a portion of one or more constant region domains are deleted or altered so as to provide the desired biochemical characteristics such as reduced serum half-life. More particularly, one skilled in the art may readily isolate the genetic sequence corresponding to the variable and/or constant regions of the subject antibody and delete or alter the appropriate nucleotides to provide modified antibodies for use as monomeric subunits in accordance with the instant invention. It will further be appreciated that compatible modified antibodies may be expressed and produced on a clinical or commercial scale using well-established protocols.

In selected embodiments, modified antibodies useful in the present invention will be derived from known antibodies to antigens associated with neoplasms or immune disorders (e.g. autoantigens). This may readily be accomplished by obtaining either the nucleotide or amino acid sequence of the parent antibody and engineering the modifications as discussed herein. For other embodiments it may be desirable to only use the antigen binding region (e.g., variable region or complementary determining regions) of the known antibody and combine them with a modified constant region to produce the desired modified antibodies that may then be used to assemble the disclosed constructs. Compatible single chain monomeric subunits may be generated in a similar manner. In any event, it will be appreciated that the antibodies of the present invention may also be engineered to improve affinity or reduce immunogenicity as is common in the art. For example, the antibodies of the present invention may be derived or fabricated from antibodies that have been humanized or chimerized. Thus, antibodies consistent with present invention may be derived or assembled from and/or comprise naturally occurring murine, primate (including human) or other mammalian monoclonal antibodies, chimeric antibodies, humanized antibodies, primatized antibodies, bispecific antibodies or single chain antibody constructs as well as immunoreactive fragments of each type.

As alluded to above, previously reported antibodies that react with tumor associated antigens may be altered as described herein to provide the antibodies of the present invention. Exemplary antibodies that may be used to provide antigen binding regions for, generate or derive the disclosed antibodies include, but are not limited to Y2B8 and C2B8 (Zevalin™ & Rituxan®, IDEC Pharmaceuticals Corp., San Diego), Lym 1 and Lym 2 (Techniclone), LL2 (Immunomedics Corp., New Jersey), HER2 (Herceptin®, Genentech Inc., South San Francisco), B1 (Bexxar®, Coulter Pharm., San Francisco), Campath® (Millennium Pharmaceuticals, Cambridge) MB1, BH3, B4, B72.3 (Cytogen Corp.), CC49 (National Cancer Institute) and 5E10 (University of Iowa). In preferred embodiments, the antibodies of the present invention will bind to the same tumor associated antigens as the antibodies enumerated immediately above. In particularly preferred embodiments, the antibodies will be derived from or bind the same antigens as Y2B8, C2B8, CC49 and C5E10 and, even more preferably, will comprise domain deleted antibodies (i.e., ΔC_{H}2 antibodies).

In a first preferred embodiment, the antibody will bind to the same tumor associated antigen as Rituxan®. Rituxan (also known as, IDEC-C2B8 and C2B8) was the first FDA-approved monoclonal antibody for treatment of human B-cell lymphoma (see U.S. Pat. Nos. 5,843,439; 5,776,456 and 5,736,137 each of which is incorporated herein by reference). Y2B8 is the murine parent of C2B8. Rituxan is a chimeric, anti-CD20 monoclonal antibody which is growth inhibitory and reportedly sensitizes certain lymphoma cell lines for apoptosis by chemotherapeutic agents in vitro. The antibody efficiently binds human complement, has strong FcR binding, and can effectively kill human lymphocytes in vitro via both complement dependent (CDC) and antibody-dependent (ADCC) mechanisms (Reff et al., Blood 83: 435-445 (1994)). Those skilled in the art will appreciate that variants (homodimers or heterodimers) of C2B8 or Y2B8, modified according to the instant disclosure, may be used in conjugated or unconjugated forms to effectively treat patients presenting with CD20+ malignancies. More generally, it must be reiterated that the modified antibodies disclosed herein may be used in either a “naked” or unconjugated state or conjugated to a cytotoxic agent to effectively treat any one of a number of disorders.

In other preferred embodiments of the present invention, the antibody will be derived from, or bind to, the same tumor associated antigen as CC49. As previously alluded to, CC49 binds human tumor associated antigen TAG-72 which is associated with the surface of certain tumor cells of human origin, specifically the LS174T tumor cell line. LS174T [American Type Culture Collection (herein ATCC) No. CL 188] is a variant of the LS180 (ATCC No. CL 187) colon adenocarcinoma line.

It will further be appreciated that numerous murine monoclonal antibodies have been developed which have binding specificity for TAG-72. One of these monoclonal antibodies, designated B72.3, is a murine IgGI produced by hybridoma B72.3 (ATCC No. HB-8108). B72.3 is a first generation monoclonal antibody developed using a human breast carcinoma extract as the immunogen (see Colcher et al., Proc. Natl. Acad. Sci. (USA), 78:3199-3203 (1981); and U.S. Pat. Nos. 4,522,918 and 4,612,282 each of which is incorporated herein by reference). Other monoclonal antibodies directed against TAG-72 are designated “CC” (for colon cancer). As described by Schlom et al. (U.S. Pat. No. 5,512,443 which is incorporated herein by reference) CC monoclonal antibodies are a family of second generation murine monoclonal antibodies that were prepared using TAG-72 purified with B72.3. Because of their relatively good binding affinities to TAG-72, the following CC antibodies have been deposited at the ATCC, with restricted access having been requested: CC49 (ATCC No. HB 9459); CC 83 (ATCC No. HB 9453); CC46 (ATCC No. HB 9458); CC92 (ATTCC No. HB 9454); CC30 (ATCC No. HB 9457); CC11 (ATCC No. 9455); and CC15 (ATCC No. HB 9460). U.S. Pat. No. 5,512,443 further teaches that the disclosed antibodies may be altered into their chimeric form by substituting, e.g., human constant regions (Fc) domains for mouse constant regions by recombinant DNA techniques known in the art. Besides disclosing murine and chimeric anti-TAG-72 antibodies, Schlom et al. have also produced variants of a humanized CC49 antibody as disclosed in PCT/US99/25552 and single chain constructs as disclosed in U.S. Pat. No. 5,892,019 each of which is also incorporated herein by reference. Those skilled in the art will appreciate that each of the foregoing antibodies, constructs or recombinants, and variations thereof, may be modified and used to provide antibodies in accordance with the present invention.

Besides the anti-TAG-72 antibodies discussed above, various groups have also reported the construction and partial characterization of domain-deleted CC49 and B72.3 antibodies (e.g., Calvo et al. Cancer Biotherapy, 8(1):95-109 (1993), Slavin-Chiorini et al. Int. J. Cancer 53:97-103 (1993) and Slavin-Chiorini et al. Cancer. Res. 55:5957-5967 (1995)). It should be appreciated that the disclosed constructs may be modified and used to provide antibodies that are compatible with the methods and compositions of the present invention.

Still other preferred embodiments of the present invention comprise modified antibodies that are derived from or bind to the same tumor associated antigen as C5E10. As set forth in co-pending application U.S. Ser. No. 09/104,717, C5E10 is an antibody that recognizes a glycoprotein determinant of approximately 115 kDa that appears to be specific to prostate tumor cell lines (e.g. DU145, PC3, or ND1). Thus, in conjunction with the present invention, modified antibodies (e.g. C_{H}2 domain-deleted antibodies) that specifically bind to the same tumor associated antigen recognized by C5E10 antibodies could be produced, assemble to form modified antibodies and used in a conjugated or unconjugated form for the treatment of neoplastic disorders. In particularly preferred embodiments, the modified antibody will be derived or comprise all or part of the antigen binding region of the C5E10 antibody as secreted from the hybridoma cell line having ATCC accession No. PTA-865. The resulting modified antibody could then be conjugated to a radionuclide as described below and administered to a patient suffering from prostate cancer in accordance with the methods herein.

In addition to the antibodies discussed above, it may be desirable to provide assemblies comprising modified antibodies derived from or comprising antigen binding regions of novel antibodies generated using immunization coupled with common immunological techniques. Using art recognized protocols, antibodies are preferably raised in mammals by multiple subcutaneous or intraperitoneal injections of the relevant antigen (e.g., purified tumor associated antigens or cells or cellular extracts comprising such antigens) and an adjuvant. This immunization typically elicits an immune response that comprises production of antigen-reactive antibodies from activated splenocytes or lymphocytes. While the resulting antibodies may be harvested from the serum of the animal to provide polyclonal preparations, it is often desirable to isolate individual lymphocytes from the spleen, lymph nodes or peripheral blood, to provide homogenous preparations of monoclonal antibodies (MAbs). Preferably, the lymphocytes are obtained from the spleen.

In this well known process (Kohler et al., Nature, 256:495(1975)) the relatively short-lived, or mortal, lymphocytes from a mammal which has been injected with antigen are fused with an immortal tumor cell line (e.g. a myeloma cell line), thus producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They therefore produce antibodies which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by immunoprecipitation or by an in vitro assay, such as a radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, pp 59-103 (Academic Press, 1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

In other compatible embodiments, DNA encoding the desired monoclonal antibodies may be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA may be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins. More particularly, the isolated DNA (which may be modified as described herein) may be used to clone constant and variable region sequences for the manufacture antibodies as described in Newman et al., U.S. Pat. No.. 5,658,570, filed Jan. 25, 1995, which is incorporated by reference herein. Essentially, this entails extraction of RNA from the selected cells, conversion to cDNA, and amplification thereof by PCR using Ig specific primers. Suitable primers for this purpose are also described in U.S. Pat. No. 5,658,570. As will be discussed in more detail below, transformed cells expressing the desired antibody may be grown up in relatively large quantities to provide clinical and commercial supplies of the immunoglobulin.

Those skilled in the art will also appreciate that DNA encoding antibodies or antibody fragments may also be derived from antibody phage libraries as set forth, for example, in EP 368 684 B1 and U.S. Pat. No. 5,969,108 each of which is incorporated herein by reference. Several publications (e.g., Marks et al. Bio/Technology 10:779-783 (1992)) have described the production of high affinity human antibodies by chain shuffling, as well as combinatorial infection and in vivo recombination as a strategy for constructing large phage libraries. Such procedures provide viable alternatives to traditional hybridoma techniques for the isolation and subsequent cloning of monoclonal antibodies and, as such, are clearly within the purview of the instant invention.

Yet other embodiments of the present invention comprise the generation of substantially human antibodies in transgenic animals (e.g., mice) that are incapable of endogenous immunoglobulin production (see e.g., U.S. Pat. Nos. 6,075,181, 5,939,598, 5,591,669 and 5,589,369 each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array in such germ line mutant mice will result in the production of human antibodies upon antigen challenge. Another preferred means of generating human antibodies using SCID mice is disclosed in commonly-owned, U.S. Pat. No. 5,811,524 which is incorporated herein by reference. It will be appreciated that the genetic material associated with these human antibodies may also be isolated and manipulated as described herein.

Yet another highly efficient means for generating recombinant antibodies is disclosed by Newman, Biotechnology, 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

As is apparent from the instant specification, genetic sequences useful for producing the antibodies of the present invention may be obtained from a number of different sources. For example, as discussed extensively above, a variety of human antibody genes are available in the form of publicly accessible deposits. Many sequences of antibodies and antibody-encoding genes have been published and suitable antibody genes can be synthesized from these sequences much as previously described. Alternatively, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

It will further be appreciated that the scope of this invention further encompasses all alleles, variants and mutations of the DNA sequences described herein.

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligodT cellulose. Techniques suitable to these purposes are familiar in the art and are described in the foregoing references.

cDNAs that encode the light and the heavy chains of the antibody may be made, either simultaneously or separately, using reverse transcriptase and DNA polymerase in accordance with well known methods. It may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes.

DNA, typically plasmid DNA, may be isolated from the cells as described herein, restriction mapped and sequenced in accordance with standard, well known techniques set forth in detail in the foregoing references relating to recombinant DNA techniques. Of course, the DNA may be modified according to the present invention at any point during the isolation process or subsequent analysis.

Preferred antibody sequences are disclosed herein. Oligonucleotide synthesis techniques compatible with this aspect of the invention are well known to the skilled artisan and may be carried out using any of several commercially available automated synthesizers. In addition, DNA sequences encoding several types of heavy and light chains set forth herein can be obtained through the services of commercial DNA synthesis vendors. The genetic material obtained using any of the foregoing methods may then be altered or modified to provide antibodies compatible with the present invention.

While a variety of different types of antibodies may be obtained and modified according to the instant invention, modified antibodies used to assemble the constructs of the instant invention will share various common traits. To that end, the term “immunoglobulin” shall be held to refer to a combination of two heavy and two light chains (H₂L₂) whether or not it possesses any relevant specific immunoreactivity. “Antibodies” refers to such assemblies which have significant known specific immunoreactive activity to an antigen (e.g. a tumor associated antigen), comprising light and heavy chains, with or without covalent linkage between them. As discussed above, “modified antibodies” according to the present invention are held to mean immunoglobulins, antibodies, or immunoreactive fragments or recombinants thereof, in which at least a fraction of one or more of the constant region domains has been deleted or otherwise altered so as to provide desired biochemical characteristics such as the ability to non-covalently dimerize, increased tumor localization or reduced serum half-life when compared with a whole, unaltered antibody of approximately the same immunogenicity. For the purposes of the instant application, immunoreactive single chain antibody constructs having altered or omitted constant region domains may be considered to be modified antibodies. As discussed above, preferred modified antibodies or domain deleted antibodies of the present invention have at least a portion of one of the constant domains deleted. More preferably, one entire domain of the constant region of the modified antibody will be deleted and even more preferably the entire C_{H}2 domain will be deleted.

Basic immunoglobulin structures in vertebrate systems are relatively well understood. As will be discussed in more detail below, the generic term “immunoglobulin” comprises five distinct classes of antibody that can be distinguished biochemically. While all five classes are clearly within the scope of the present invention, the following discussion will generally be directed to the class of IgG molecules. With regard to IgG, immunoglobulins comprise two identical light polypeptide chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

More specifically, both the light and heavy chains are divided into regions of structural and functional homology. The terms “constant” and “variable” are used functionally. In this regard, it will be appreciated that the variable domains of both the light (V_{L}) and heavy (V_{H}) chains determine antigen recognition and specificity. Conversely, the constant domains of the light chain (C_{L}) and the heavy chain (C_{H}1, C_{H}2 or C_{H}3) confer important biological properties such as secretion, transplacental mobility, Fc receptor binding, complement binding, and the like. By convention the numbering of the constant region domains increases as they become more distal from the antigen binding site or amino-terminus of the antibody. Thus, the C_{H}3 and C_{L} domains actually comprise the carboxy-terminus of the heavy and light chains respectively.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages” when the immunoglobulins are generated either by hybridomas, B cells or genetically engineered host cells. In the heavy chain, the amino acid sequences run from an N-terminus at the forked ends of the Y configuration to the C-terminus at the bottom of each chain. At the N-terminus is a variable region and at the C-terminus is a constant region. Those skilled in the art will appreciate that heavy chains are classified as gamma, mu, alpha, delta, or epsilon, (γ, μ, α, δ, ε) with some subclasses among them. It is the nature of this chain that determines the “class” of the antibody as IgA, IgD, IgE IgG, or IgM. The immunoglobulin subclasses (isotypes) e.g. IgG₁, IgG₂, IgG₃, IgG₄, IgA₁, etc. are well characterized and are known to confer functional specialization. Modified versions of each of these classes and isotypes are readily discernable to the skilled artisan in view of the instant disclosure and, accordingly, are within the purview of the instant invention.

As indicated above, the variable region allows the antibody to selectively recognize and specifically bind epitopes on immunoreactive antigens. That is, the V_{L} domain and V_{H} domain of an antibody combine to form the variable region that defines a three dimensional antigen binding site. This quaternary antibody structure provides for an antigen binding site present at the end of each arm of the Y.

The six CDRs present on each monomeric antibody (H₂L₂) are short, non-contiguous sequences of amino acids that are specifically positioned to form the antigen binding site as the antibody assumes its three dimensional configuration in an aqueous environment. The remainder of the heavy and light variable domains show less inter-molecular variability in amino acid sequence and are termed the framework regions. The framework regions largely adopt a β-sheet conformation and the CDRs form loops connecting, and in some cases forming part of, the β-sheet structure. Thus, these framework regions act to form a scaffold that provides for positioning the six CDRs in correct orientation by inter-chain, non-covalent interactions. In any event, the antigen binding site formed by the positioned CDRs defines a surface complementary to the epitope on the immunoreactive antigen. This complementary surface promotes the non-covalent binding of the antibody to the immunoreactive antigen epitope.

For the purposes of the present invention, it should be appreciated that disclosed modified antibodies may comprise any type of variable region that provides for the association of the antibody with the selected antigen. In this regard, the variable region may comprise or be derived from any type of mammal that can be induced to mount a humoral response and generate immunoglobulins against the desired antigen. As such, the variable region of the modified antibodies may be, for example, of human, murine, non-human primate (e.g. cynomolgus monkeys, macaques, etc.) or lupine origin. In particularly preferred embodiments both the variable and constant regions of compatible modified antibodies are human. In other selected embodiments the variable regions of compatible antibodies (usually derived from a non-human source) may be engineered or specifically tailored to improve the binding properties or reduce the immunogenicity of the molecule. In this respect, variable regions useful in the present invention may be humanized or otherwise altered through the inclusion of imported DNA or amino acid sequences.

For the purposes of the instant application the term “humanized antibody” shall mean an antibody derived from a non-human antibody, typically a murine antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81: 6851-5 (1984); Morrison et al., Adv. Immunol. 44: 65-92 (1988); Verhoeyen et al., Science 239: 1534-1536 (1988); Padlan, Molec. Immun. 28: 489-498 (1991); Padlan, Molec. Immun. 31: 169-217 (1994), and U.S. Pat. Nos. 5,585,089, 5,693,761 and 5,693,762 all of which are hereby incorporated by reference in their entirety.

Those skilled in the art will appreciate that the technique set forth in option (a) above will produce “classic” chimeric antibodies. In the context of the present application the term “chimeric antibodies” will be held to mean any antibody wherein the immunoreactive region or site is obtained or derived from a first species and the constant region (which may be intact, partial or modified in accordance with the instant invention) is obtained from a second species. In preferred embodiments the antigen binding region or site will be from a non-human source (e.g. mouse) and the constant region is human. While the immunogenic specificity of the variable region is not generally affected by its source, a human constant region is less likely to elicit an immune response from a human subject than would the constant region from a non-human source.

Preferably, the variable domains in both the heavy and light chains are altered by at least partial replacement of one or more CDRs and, if necessary, by partial framework region replacement and sequence changing. Although the CDRs may be derived from an antibody of the same class or even subclass as the antibody from which the framework regions are derived, it is envisaged that the CDRs will be derived from an antibody of different class and preferably from an antibody from a different species. It must be emphasized that it may not be necessary to replace all of the CDRs with the complete CDRs from the donor variable region to transfer the antigen binding capacity of one variable domain to another. Rather, it may only be necessary to transfer those residues that are necessary to maintain the activity of the antigen binding site. Given the explanations set forth in U. S. Pat. Nos. 5,585,089, 5,693,761 and 5,693,762, it will be well within the competence of those skilled in the art, either by carrying out routine experimentation or by trial and error testing to obtain a functional antibody with reduced immunogenicity.

Alterations to the variable region notwithstanding, those skilled in the art will appreciate that modified antibodies compatible with the instant invention will comprise antibodies, or immunoreactive fragments thereof, in which at least a fraction of one or more of the constant region domains has been deleted or otherwise altered so as to provide desired biochemical characteristics such as increased tumor localization or reduced serum half-life when compared with an antibody of approximately the same immunogenicity comprising a native or unaltered constant region. In preferred embodiments, the constant region of the modified antibodies will comprise a human constant region. Modifications to the constant region compatible with the instant invention comprise additions, deletions or substitutions of one or more amino acids in one or more domains. That is, the modified antibodies disclosed herein may comprise alterations or modifications to one or more of the three heavy chain constant domains (C_{H}1, C_{H}2 or C_{H}3) and/or to the light chain constant domain (C_{L}). As will be discussed in more detail below and shown in the examples, preferred embodiments of the invention comprise modified constant regions wherein one or more domains are partially or entirely deleted (“domain deleted antibodies”). In especially preferred embodiments compatible modified antibodies will comprise domain deleted constructs or variants wherein the entire C_{H}2 domain has been removed (ΔC_{H}2 constructs). For other preferred embodiments a short amino acid spacer may be substituted for the deleted domain to provide flexibility and freedom of movement for the variable region.

As previously indicated, the subunit structures and three dimensional configuration of the constant regions of the various immunoglobulin classes are well known. For example, the C_{H}2 domain of a human IgG Fc region usually extends from about residue 231 to residue 340 using conventional numbering schemes. The C_{H}2 domain is unique in that it is not closely paired with another domain. Rather, two N-linked branched carbohydrate chains are interposed between the two C_{H}2 domains of an intact native IgG molecule. It is also well documented that the C_{H}3 domain extends from the C_{H}2 domain to the C-terminal of the IgG molecule and comprises approximately 108 residues while the hinge region of an IgG molecule joins the C_{H}2 domain with the C_{H}1 domain. This hinge region encompasses on the order of 25 residues and is flexible, thereby allowing the two N-terminal antigen binding regions to move independently.

Besides their configuration, it is known in the art that the constant region mediates several effector functions. For example, binding of the C1 component of complement to antibodies activates the complement system. Activation of complement is important in the opsonisation and lysis of cell pathogens. The activation of complement also stimulates the inflammatory response and may also be involved in autoimmune hypersensitivity. Further, antibodies bind to cells via the Fc region, with a Fc receptor site on the antibody Fc region binding to a Fc receptor (FcR) on a cell. There are a number of Fc receptors which are specific for different classes of antibody, including IgG (gamma receptors), IgE (eta receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. Although various Fc receptors and receptor sites have been studied to a certain extent, there is still much which is unknown about their location, structure and functioning.

Moreover, while not limiting the scope of the present invention, it is believed that antibodies comprising constant regions modified as described herein provide for altered effector functions that, in turn, affect the biological profile of the administered antibody. For example, the deletion or inactivation (through point mutations or other means) of a constant region domain may reduce Fc receptor binding of the circulating modified antibody thereby increasing tumor localization. In other cases it may be that constant region modifications consistent with the instant invention moderate compliment binding and thus reduce the serum half life and nonspecific association of a conjugated cytotoxin. Yet other modifications of the constant region may be used to eliminate disulfide linkages or oligosaccharide moities that allow for enhanced localization due to increased antigen specificity or antibody flexibility. More generally, those skilled in the art will realize that antibodies modified as described herein may exert a number of subtle effects that may or may not be readily appreciated. However the resulting physiological profile, bioavailability and other biochemical effects of the modifications, such as tumor localization and serum half-life, may easily be measured and quantified using well know immunlogical techniques without undue experimentation.

Similarly, modifications to the constant region in accordance with the instant invention may easily be made using well known biochemical or molecular engineering techniques well within the purview of the skilled artisan.

Following manipulation of the isolated genetic material to provide modified antibodies as set forth above, the genes are typically inserted in an expression vector for introduction into host cells that may be used to produce the desired quantity of modified antibody that, in turn, provides the claimed constructs.

The term “vector” or “expression vector” is used herein for the purposes of the specification and claims, to mean vectors used in accordance with the present invention as a vehicle for introducing into and expressing a desired gene in a cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

In particularly preferred embodiments the cloned variable region genes are inserted into an expression vector along with the heavy and light chain constant region genes (preferably human) modified as discussed above. Preferably, this is effected using a proprietary expression vector of IDEC, Inc., referred to as NEOSPLA. This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. As seen in the examples below, this vector has been found to result in very high level expression of antibodies upon incorporation of variable and constant region genes, transfection in CHO cells, followed by selection in G418 containing medium and methotrexate amplification. This vector system is substantially disclosed in commonly assigned U.S. Pat. Nos. 5,736,137 and 5,658,570, each of which is incorporated by reference in its entirety herein. This system provides for high expression levels, i.e., >30 pg/cell/day.

In other preferred embodiments the modified antibodies of the instant invention may be expressed using polycistronic constructs such as those disclosed in copending U.S. provisional application No. 60/331,481 filed Nov. 16, 2001 and incorporated herein in its entirety. In these novel expression systems, multiple gene products of interest such as heavy and light chains of antibodies may be produced from a single polycistronic construct. These systems advantageously use an internal ribosome entry site (IRES) to provide relatively high levels of modified antibodies in eukaryotic host cells. Compatible IRES sequences are disclosed in U.S. Pat. No. 6,193,980 which is also incorporated herein. Those skilled in the art will appreciate that such expression systems may be used to effectively produce the full range of modified antibodies disclosed in the instant application.

More specifically, the antibodies of the present invention will be advantageously produced through a novel expression system for producing multiple gene products of interest from a single polycistronic construct. In a preferred embodiment, the expression system produces antibodies in eukaryotic cells, preferably mammalian cells such as Chinese hamster ovary (CHO) cells, baby hamster kidney (BHK) cells, fibroblast cell lines, myeloma cells. Preferably, CHO cells are employed as hosts for an expression system comprising a polycistron comprising DNA coding GnYIII and, in the 5′ to 3′ orientation, at least the following sequences: a strong eukaryotic promoter sequence such as CMV, SV40 early or actin promoter sequences, preferably CMV; a DNA sequence encoding an antibody light chain and, preferably at its 5′ end, a eukaryotic secreting leader sequence; an internal ribosome entry site (IRES), preferably that of a cardiovirus, poliovirus or herpes virus, positioned to follow the antibody light chain sequence; at least one DNA sequence encoding an antibody heavy chain, each heavy chain sequence preferably being preceded by a eukaryotic secreting leader sequence, and flanked by a start and a stop codon, wherein each DNA encoding an antibody heavy chain is separated from a subsequent heavy chain sequence by an IRES, and wherein the ultimate antibody heavy chain coding sequence comprises a poly A sequence at its 3′ terminus.

As noted herein, the eukaryotic cell preferably comprises a mammalian cell and more preferably a CHO cell. In a preferred embodiment, the promoter is the CMV promoter, and the IRES is derived from a cardiovirus such as Encephalomyocarditis virus, Mengo virus, Mous-Elberfiell virus, MM virus, and Columbia SK virus, most preferably human encephalomyocarditis virus (hEMCV).

The inventive polycistron preferably comprises one or two antibody heavy chain coding sequences. However, polycistron combinations including 3 or 4 gene sequences, for example, one light chain and three heavy chains, are contemplated. Additionally, the subject polycistron will preferably comprise the poly A sequence of the bovine growth hormone (bGH) gene. The polycistron system may be used in homologous recombination with IRES.

In a preferred embodiment the inventive polycistron comprises one or two copies of the heavy chain coding sequence dependent upon the stoichiometry of gene expression. In this regard, it is well known that in polycistronic expression systems, the second gene is expressed at lesser efficiency than the first gene. Accordingly, the inventive polycistron, in which the first cistron encodes an antibody light chain, may encompass a second cistron encoding two or more heavy chain coding sequences, if deemed necessary, to facilitate sufficient expression of the heavy chain relative to the light chain. In general, it is preferred that the heavy chain be expressed at levels which are at least equivalent to levels observed with non-polycistronic co-expression of the heavy and light chains.

It is permissible, and in fact desirable, that more of the antibody light chain is expressed in comparison with the heavy chain, as this is analogous to what occurs in endogenous cells of a mammal. Disparate expression levels exist because the light chain is instrumental in directing the appropriate assembly of the antibody heavy and light chains, and excessive unpaired heavy chain is thought to induce cell toxicity. The light chain is also critical in directing folding of the assembled antibody heavy and light chains to produce a functional (antigen-binding) antibody in the endoplasmic reticulum.

However, levels of the heavy chain must not be de minimus, and should be present in sufficient ratios with respect to light chains to enable the genesis of functional, secretable antibodies in commercially acceptable levels. Thus, it is undesirable for the heavy chain expression to be too low relative to the light chain, as underexpression results in inadequate yields of functional antibodies. For purposes of industrial utility, inadequate yields of functional antibodies render an expression system commercially non-viable, and makes the recovery of complete antibody molecules from batch cultures difficult to achieve. Preferably, functional antibody is recovered from cultured cells at an amount ranging from about 10-50 picograms/cell.

With respect to the above, it is generally unpredictable whether a given polycistronic expression system will result in adequate levels of antibody production relative to other expression systems. This unpredictability arises because, in some instances, the second desired gene in the polycistronic complex may be expressed at very low levels relative to the first gene. Therefore, preferred embodiments of polycistronic vectors should provide a ratio of antibody light chain expression to antibody heavy chain expression within the range of about 10:1 to about 1:1. Preferably, the ratio of light chain to heavy chain gene expression is from about 3:1 to about 2:1.

Initial IRES constructs were crafted to contain an antibody light chain sequence in the fist cistron, followed by two IRES-antibody CH2 domain deleted heavy chain sequence pairings, thereby ensuring sufficient heavy chain protein production to enable suitable levels of antibody to be produced and secreted from host cells. The known unpredictability of second cistron expression in polycistronic vectors prompted the construction of such a vector. Surprisingly, both heavy chain sequences could be expressed via this polycistronic vector.

The inventive polycistronic vectors enable the requisite levels of heavy and light chain expression to be achieved by judicious selection of appropriate heavy chain antibody sequences, by selection of an efficient IRES, such as that of hEMCV, or by the incorporation of multiple copies of the antibody heavy chain genes. Still further, the DNA corresponding to the 5′ end of the heavy chain gene may be modified by site specific mutagenesis such that the coding structure remains unaltered around the ATG codon, typically the first 10 codons. In this manner, the expression levels of different heavy chain coding sequences compound, thereby selecting for a heavy chain DNA that provides optimal yields of antibody heavy chain molecules relative to antibody light chain molecules.

The heavy chain yield will be less than the light chain yield, as is the typical expression relationship in the intact cell. The light chain yield to heavy chain yield ratio will be sufficient to enable protein secretion and folding. The ratio of the light chain to heavy chain expression may be varied by, for example, increasing the number of IRES-linked downstream gene sequences following the light chain sequence of the first cistron. A particular IRES and expression cell combination may be selected to optimally increase the amount of second cistron expression in a system.

More generally, once the vector or DNA sequence encoding the antibody has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Most preferably, plasmid introduction into the host is via electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

As used herein, the term “transformation” shall be used in a broad sense to refer to any introduction of DNA into a recipient host cell that changes the genotype and consequently results in a change in the recipient cell.

Along those same lines, “host cells” refers to cells that have been transformed with vectors constructed using recombinant DNA techniques and encoding at least one heterologous gene. As defined herein, antibodies or modifications thereof produced by a host cell that is, by virtue of this transformation, recombinant. In descriptions of processes for isolation of antibodies from recombinant hosts, the terms “cell” and “cell culture” are used interchangeably to denote the source of antibody unless it is clearly specified otherwise. In other words, recovery of antibody from the “cells” may mean either from spun down whole cells, or from the cell culture containing both the medium and the suspended cells.

The host cell line used for protein expression is most preferably of mammalian origin; those skilled in the art are credited with ability to preferentially determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3.times.63-Ag3.653 (mouse myeloma), BFA-1c1 BPT (bovine endothelial cells), RAJI (human lymphocyte) and 293 (human kidney). CHO cells are particularly preferred. Host cell lines are typically available from commercial services, the American Tissue Culture Collection or from published literature.

In vitro production allows scale-up to give large amounts of the desired monomeric subunit and, by extension, constructs. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. As previously described, at least some of the monomeric subunits spontaneously associate non-covalently to form antibodies. For isolation and recovery of the antibodies, the immunoglobulins in the culture supernatants may first be concentrated, e.g. by precipitation with ammonium sulphate, dialysis against hygroscopic material such as PEG, filtration through selective membranes, or the like.

Modified antibody genes can also be expressed non-mammalian cells such as bacteria or yeast. In this regard it will be appreciated that various unicellular non-mammalian microorganisms such as bacteria can also be transformed; i.e. those capable of being grown in cultures or fermentation. Bacteria, which are susceptible to transformation, include members of the enterobacteriaceae, such as strains of Escherichia coli; Salmonella; Bacillaceae, such as Bacillus subtilis; Pneumococcus; Streptococcus, and Haemophilus influenzae. It will further be appreciated that, when expressed in bacteria, the immunoglobulin heavy chains and light chains typically become part of inclusion bodies. The chains then must be isolated, purified and then assembled into functional monomeric subunits.

In addition to prokaryates, eukaryotic microbes may also be used. Saccharomyces cerevisiae, or common baker's yeast, is the most commonly used among eukaryotic microorganisms although a number of other strains are commonly available.

For expression in Saccharomyces, the plasmid YRp7, for example, (Stinchcomb et al., Nature, 282:39 (1979); Kingsman et al., Gene, 7:141 (1979); Tschemper et al., Gene, 10:157 (1980)) is commonly used. This plasmid already contains the trpl gene which provides a selection marker for a mutant strain of yeast lacking the ability to grow in tryptophan, for example ATCC No. 44076 or PEP4-1 (Jones, Genetics, 85:12 (1977)). The presence of the trpl lesion as a characteristic of the yeast host cell genome then provides an effective environment for detecting transformation by growth in the absence of tryptophan.

Regardless of how clinically useful quantities are obtained, the antibodies of the present invention may be used in any one of a number of conjugated (i.e. an immunoconjugate) or unconjugated forms. In particular, the antibodies of the present invention may be conjugated to cytotoxins such as radioisotopes, therapeutic agents, cytostatic agents, biological toxins or prodrugs. Alternatively, the antibodies of the instant invention may be used in a nonconjugated or original form to harness the subject's natural defense mechanisms to eliminate the malignant cells. In particularly preferred embodiments, the modified antibodies may be conjugated to radioisotopes, such as ⁹⁰Y, ¹²⁵I, ¹³¹I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re using anyone of a number of well known chelators or direct labeling. In other embodiments, the disclosed compositions may comprise modified antibodies coupled to drugs, prodrugs or biological response modifiers such as methotrexate, adriamycin, and lymphokines such as interferon. Still other embodiments of the present invention comprise the use of modified antibodies conjugated to specific biotoxins such as ricin or diptheria toxin. In yet other embodiments the modified antibodies may be complexed with other immunologically active ligands (e.g. antibodies or fragments thereof) wherein the resulting molecule binds to both the neoplastic cell and an effector cell such as a T cell. The selection of which conjugated or unconjugated modified antibody to use will depend of the type and stage of cancer, use of adjunct treatment (e.g., chemotherapy or external radiation) and patient condition. It will be appreciated that one skilled in the art could readily make such a selection in view of the teachings herein.

As used herein, “a cytotoxin or cytotoxic agent” means any agent that is detrimental to the growth and proliferation of cells and may act to reduce, inhibit or destroy a cell or malignancy when exposed thereto. Exemplary cytotoxins include, but are not limited to, radionuclides, biotoxins, enzymatically active toxins, cytostatic or cytotoxic therapeutic agents, prodrugs, immunologically active ligands and biological response modifiers such as cytokines. As will be discussed in more detail below, radionuclide cytotoxins are particularly preferred for use in the instant invention. However, any cytotoxin that acts to retard or slow the growth of immunoreactive cells or malignant cells or to eliminate these cells and may be associated with the antibodies disclosed herein is within the purview of the present invention.

It will be appreciated that, in previous studies, anti-tumor antibodies labeled with these isotopes have been used successfully to destroy cells in solid tumors as well as lymphomas/leukemias in animal models, and in some cases in humans. The radionuclides act by producing ionizing radiation which causes multiple strand breaks in nuclear DNA, leading to cell death. The isotopes used to produce therapeutic conjugates typically produce high energy α- or β-particles which have a short path length. Such radionuclides kill cells to which they are in close proximity, for example neoplastic cells to which the conjugate has attached or has entered. They have little or no effect on non-localized cells. Radionuclides are essentially non-immunogenic.

It will be appreciated that, in previous studies, anti-tumor antibodies labeled with isotopes have been used successfully to destroy cells in solid tumors as well as lymphomas/leukemias in animal models, and in some cases in humans. The radionuclides act by producing ionizing radiation which causes multiple strand breaks in nuclear DNA, leading to cell death. The isotopes used to produce therapeutic conjugates typically produce high energy α-, γ- or β-particles which have a therapeutically effective path length. Such radionuclides kill cells to which they are in close proximity, for example neoplastic cells to which the conjugate has attached or has entered. They generally have little or no effect on non-localized cells. Radionuclides are essentially non-immunogenic.

With respect to the use of radiolabeled conjugates in conjunction with the present invention, the modified antibodies may be directly labeled (such as through iodination) or may be labeled indirectly through the use of a chelating agent. As used herein, the phrases “indirect labeling” and “indirect labeling approach” both mean that a chelating agent is covalently attached to an antibody and at least one radionuclide is associated with the chelating agent. Such chelating agents are typically referred to as bifunctional chelating agents as they bind both the polypeptide and the radioisotope. Particularly preferred chelating agents comprise 1-isothiocycmatobenzyl-3-methyldiothelene triaminepentaacetic acid (“MX-DTPA”) and cyclohexyl diethylenetriamine pentaacetic acid (“CHX-DTPA”) derivatives. Other chelating agents comprise P-DOTA and EDTA derivatives. Particularly preferred radionuclides for indirect labeling include ¹¹¹In and ⁹⁰Y.

As used herein, the phrases “direct labeling” and “direct labeling approach” both mean that a radionuclide is covalently attached directly to a antibody (typically via an amino acid residue). More specifically, these linking technologies include random labeling and site-directed labeling. In the latter case, the labeling is directed at specific sites on the antibody, such as the N-linked sugar residues present only on the Fc portion of the conjugates. Further, various direct labeling techniques and protocols are compatible with the instant invention. For example, Technetium-99m labeled antibodies may be prepared by ligand exchange processes, by reducing pertechnate (TcO₄⁻) with stannous ion solution, chelating the reduced technetium onto a Sephadex column and applying the antibodies to this column, or by batch labeling techniques, e.g. by incubating pertechnate, a reducing agent such as SnCl₂, a buffer solution such as a sodium-potassium phthalate-solution, and the antibodies. In any event, preferred radionuclides for directly labeling antibodies are well known in the art and a particularly preferred radionuclide for direct labeling is ¹³¹I covalently attached via tyrosine residues. Modified antibodies according to the invention may be derived, for example, with radioactive sodium or potassium iodide and a chemical oxidizing agent, such as sodium hypochlorite, chloramine T or the like, or an enzymatic oxidizing agent, such as lactoperoxidase, glucose oxidase and glucose. However, for the purposes of the present invention, the indirect labeling approach is particularly preferred.

Patents relating to chelators and chelator conjugates are known in the art. For instance, U.S. Pat. No. 4,831,175 of Gansow is directed to polysubstituted diethylenetriaminepentaacetic acid chelates and protein conjugates containing the same, and methods for their preparation. U.S. Pat. Nos. 5,099,069, 5,246,692, 5,286,850, 5,434,287 and 5,124,471 of Gansow also relate to polysubstituted DTPA chelates. These patents are incorporated herein in their entirety. Other examples of compatible metal chelators are ethylenediaminetetraacetic acid (EDTA), diethylenetriaminepentaacetic acid (DPTA), 1,4,8,11-tetraazatetradecane, 1,4,8,11-tetraazatetradecane-1,4,8,11-tetraacetic acid, 1-oxa-4,7,12,15-tetraazaheptadecane-4,7,12,15-tetraacetic acid, or the like. Cyclohexyl-DTPA or CHX-DTPA is particularly preferred and is exemplified extensively below. Still other compatible chelators, including those yet to be discovered, may easily be discerned by a skilled artisan and are clearly within the scope of the present invention.

Compatible chelators, including the specific bifunctional chelator used to facilitate chelation in co-pending application Ser. Nos. 08/475,813, 08/475,815 and 08/478,967, are preferably selected to provide high affinity for trivalent metals, exhibit increased tumor-to-non-tumor ratios and decreased bone uptake as well as greater in vivo retention of radionuclide at target sites, i.e., B-cell lymphoma tumor sites. However, other bifunctional chelators that may or may not possess all of these characteristics are known in the art and may also be beneficial in tumor therapy.

It will also be appreciated that, in accordance with the teachings herein, modified antibodies may be conjugated to different radiolabels for diagnostic and therapeutic purposes. To this end the aforementioned co-pending applications, herein incorporated by reference in their entirety, disclose radiolabeled therapeutic conjugates for diagnostic “imaging” of tumors before administration of therapeutic antibody. “In2B8” conjugate comprises a murine monoclonal antibody, 2B8, specific to human CD20 antigen, that is attached to ¹¹¹In via a bifunctional chelator, i.e., MX-DTPA (diethylenetriaminepentaacetic acid), which comprises a 1:1 mixture of 1-isothiocyanatobenzyl-3-methyl-DTPA and 1-methyl-3-isothiocyanatobenzyl-DTPA. ¹¹¹In is particularly preferred as a diagnostic radionuclide because between about 1 to about 10 mCi can be safely administered without detectable toxicity; and the imaging data is generally predictive of subsequent ⁹⁰Y-labeled antibody distribution. Most imaging studies utilize 5 mCi ¹¹¹In-labeled antibody, because this dose is both safe and has increased imaging efficiency compared with lower doses, with optimal imaging occurring at three to six days after antibody administration. See, for example, Murray, J. Nuc. Med. 26: 3328 (1985) and Carraguillo et al., J. Nuc. Med. 26: 67 (1985).

As indicated above, a variety of radionuclides are applicable to the present invention and those skilled in the art are credited with the ability to readily determine which radionuclide is most appropriate under various circumstances. For example, ¹³¹I is a well known radionuclide used for targeted immunotherapy. However, the clinical usefulness of ¹³¹I can be limited by several factors including: eight-day physical half-life; dehalogenation of iodinated antibody both in the blood and at tumor sites; and emission characteristics (e.g., large gamma component) which can be suboptimal for localized dose deposition in tumor. With the advent of superior chelating agents, the opportunity for attaching metal chelating groups to proteins has increased the opportunities to utilize other radionuclides such as ¹¹¹In and ⁹⁰Y. ⁹⁰Y provides several benefits for utilization in radioimmunotherapeutic applications: the 64 hour half-life of ⁹⁰Y is long enough to allow antibody accumulation by tumor and, unlike e.g., ¹³¹I, ⁹⁰Y is a pure beta emitter of high energy with no accompanying gamma irradiation in its decay, with a range in tissue of 100 to 1,000 cell diameters. Furthermore, the minimal amount of penetrating radiation allows for outpatient administration of ⁹⁰Y-labeled antibodies. Additionally, internalization of labeled antibody is not required for cell killing, and the local emission of ionizing radiation should be lethal for adjacent tumor cells lacking the target antigen.

Effective single treatment dosages (i.e., therapeutically effective amounts) of ⁹⁰Y-labeled modified antibodies range from between about 5 and about 75 mCi, more preferably between about 10 and about 40 mCi. Effective single treatment non-marrow ablative dosages of ¹³¹I-labeled antibodies range from between about 5 and about 70 mCi, more preferably between about 5 and about 40 mCi. Effective single treatment ablative dosages (i.e., may require autologous bone marrow transplantation) of ¹³¹I-labeled antibodies range from between about 30 and about 600 mCi, more preferably between about 50 and less than about 500 mCi. In conjunction with a chimeric antibody, owing to the longer circulating half life vis-á-vis murine antibodies, an effective single treatment non-marrow ablative dosages of iodine-131 labeled chimeric antibodies range from between about 5 and about 40 mCi, more preferably less than about 30 mCi. Imaging criteria for, e.g., the ¹¹¹In label, are typically less than about 5 mCi.

While a great deal of clinical experience has been gained with ¹³¹I and ⁹⁰Y, other radiolabels are known in the art and have been used for similar purposes. Still other radioisotopes are used for imaging. For example, additional radioisotopes which are compatible with the scope of the instant invention include, but are not limited to, ¹²³I, ¹²⁵I, ³²P, ⁵⁷Co, ⁶⁴CU, ⁶⁷CU, ⁷⁷Br, ⁸¹Rb, 81Kr, ⁸⁷Sr, ¹¹³In, ¹²⁷Cs, ¹²⁹Cs, ¹³²I, ¹⁹⁷Hg, ²⁰³Pb, ²⁰⁶Bi, 177Lu, ¹⁸⁶Re, 212Pb, ²¹²Bi, ⁴⁷Sc, ¹⁰⁵Rh ¹⁰⁹Pd, ¹⁵³Sm, ¹⁸⁸Re, ¹⁹⁹Au, ²²⁵Ac, 211At, and 213Bi. In this respect alpha, gamma and beta emitters are all compatible with in the instant invention. Further, in view of the instant disclosure it is submitted that one skilled in the art could readily determine which radionuclides are compatible with a selected course of treatment without undue experimentation. To this end, additional radionuclides which have already been used in clinical diagnosis include ¹²¹I, ¹²³I, ⁹⁹Tc, ⁴³K, ⁵²Fe, ⁶⁷Ga ⁶⁸Ga, as well as ¹¹¹In. antibodies have also been labeled with a variety of radionuclides for potential use in targeted immunotherapy Peirersz et al. Immunol. Cell Biol. 65: 111-125 (1987). These radionuclides include ¹⁸⁸Re and ¹⁸⁶Re as well as ¹⁹⁹Au and ⁶⁷Cu to a lesser extent. U.S. Pat. No. 5,460,785 provides additional data regarding such radioisotopes and is incorporated herein by reference.

In addition to radionuclides, the antibodies of the present invention may be conjugated to, or associated with, any one of a number of biological response modifiers, pharmaceutical agents, toxins or immunologically active ligands. Those skilled in the art will appreciate that these non-radioactive conjugates may be assembled using a variety of techniques depending on the selected cytotoxin. For example, conjugates with biotin are prepared e.g. by reacting the antibodies with an activated ester of biotin such as the biotin N-hydroxysuccinimide ester. Similarly, conjugates with a fluorescent marker may be prepared in the presence of a coupling agent, e.g. those listed above, or by reaction with an isothiocyanate, preferably fluorescein-isothiocyanate. Conjugates of the antibodies of the invention with cytostatic/cytotoxic substances and metal chelates are prepared in an analogous manner.

Preferred agents for use in the present invention are cytotoxic drugs, particularly those which are used for cancer therapy. Such drugs include, in general, cytostatic agents, alkylating agents, antimetabolites, anti-proliferative agents, tubulin binding agents, hormones and hormone antagonists, and the like. Exemplary cytostatics that are compatible with the present invention include alkylating substances, such as mechlorethamine, triethylenephosphoramide, cyclophosphamide, ifosfamide, chlorambucil, busulfan, melphalan or triaziquone, also nitrosourea compounds, such as carmustine, lomustine, or semustine. Other preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, adriamycin, carminomycin, daunorubicin (daunomycin), doxorubicin, aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, or podophyllotoxin derivatives such as etoposide or etoposide phosphate, melphalan, vinblastine, vincristine, leurosidine, vindesine, leurosine and the like. Still other cytotoxins that are compatible with the teachings herein include taxol, taxane, cytochalasin B, gramicidin D, ethidium bromide, emetine, tenoposide, colchicin, dihydroxy anthracin dione, mitoxantrone, procaine, tetracaine, lidocaine, propranolol, and puromycin and analogs or homologs thereof. Hormones and hormone antagonists, such as corticosteroids, e.g. prednisone, progestins, e.g. hydroxyprogesterone or medroprogesterone, estrogens, e.g. diethylstilbestrol, antiestrogens, e.g. tamoxifen, androgens, e.g. testosterone, and aromatase inhibitors, e.g. aminogluthetimide are also compatible with the teachings herein. As noted previously, one skilled in the art may make chemical modifications to the desired compound in order to make reactions of that compound more convenient for purposes of preparing conjugates of the invention.

One example of particularly preferred cytotoxins comprise members or derivatives of the enediyne family of anti-tumor antibiotics, including calicheamicin, esperamicins or dynemicins. These toxins are extremely potent and act by cleaving nuclear DNA, leading to cell death. Unlike protein toxins which can be cleaved in vivo to give many inactive but immunogenic polypeptide fragments, toxins such as calicheamicin, esperamicins and other enediynes are small molecules which are essentially non-immunogenic. These non-peptide toxins are chemically-linked to the antibodies by techniques which have been previously used to label monoclonal antibodies and other molecules. These linking technologies include site-specific linkage via the N-linked sugar residues present only on the Fc portion of the constructs. Such site-directed linking methods have the advantage of reducing the possible effects of linkage on the binding properties of the constructs.

As previously alluded to, compatible cytotoxins may comprise a prodrug. As used herein, the term “prodrug” refers to a precursor or derivative form of a pharmaceutically active substance that is less cytotoxic to tumor cells compared to the parent drug and is capable of being enzymatically activated or converted into the more active parent form. Prodrugs compatible with the invention include, but are not limited to, phosphate-containing prodrugs, thiophosphate-containing prodrugs, sulfate containing prodrugs, peptide containing prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs that can be converted to the more active cytotoxic free drug. Further examples of cytotoxic drugs that can be derivatized into a prodrug form for use in the present invention comprise those chemotherapeutic agents described above.

Among other cytotoxins, it will be appreciated that antibodies can also be associated with a biotoxin such as ricin subunit A, abrin, diptheria toxin, botulinum, cyanginosins, saxitoxin, shigatoxin, tetanus, tetrodotoxin, trichothecene, verrucologen or a toxic enzyme. Preferably, such constructs will be made using genetic engineering techniques that allow for direct expression of the antibody-toxin construct. Other biological response modifiers that may be associated with the modified antibodies of the present invention comprise cytokines such as lymphokines and interferons. In view of the instant disclosure it is submitted that one skilled in the art could readily form such constructs using conventional techniques.

Another class of compatible cytotoxins that may be used in conjunction with the disclosed antibodies are radiosensitizing drugs that may be effectively directed to tumor or immunoreactive cells. Such drugs enhance the sensitivity to ionizing radiation, thereby increasing the efficacy of radiotherapy. An antibody conjugate internalized by the tumor cell would deliver the radiosensitizer nearer the nucleus where radiosensitization would be maximal. The unbound radiosensitizer linked modified antibodies would be cleared quickly from the blood, localizing the remaining radiosensitization agent in the target tumor and providing minimal uptake in normal tissues. After rapid clearance from the blood, adjunct radiotherapy would be administered in one of three ways: 1.) external beam radiation directed specifically to the tumor, 2.) radioactivity directly implanted in the tumor or 3.) systemic radioimmunotherapy with the same targeting antibody. A potentially attractive variation of this approach would be the attachment of a therapeutic radioisotope to the radiosensitized immunoconjugate, thereby providing the convenience of administering to the patient a single drug.

Whether or not the disclosed antibodies are used in a conjugated or unconjugated form, it will be appreciated that a major advantage of the present invention is the ability to use these constructs in myelosuppressed patients, especially those who are undergoing, or have undergone, adjunct therapies such as radiotherapy or chemotherapy. That is, the beneficial delivery profile (i.e. relatively short serum dwell time, high binding affinity and enhanced localization) of the antibodies makes them particularly useful for treating patients that have reduced red marrow reserves and are sensitive to myelotoxicity. In this regard, the unique delivery profile of the antibodies make them very effective for the administration of radiolabeled conjugates to myelosuppressed cancer patients. As such, the modified antibodies are useful in a conjugated or unconjugated form in patients that have previously undergone adjunct therapies such as external beam radiation or chemotherapy. In other preferred embodiments, the antibodies (again in a conjugated or unconjugated form) may be used in a combined therapeutic regimen with chemotherapeutic agents. Those skilled in the art will appreciate that such therapeutic regimens may comprise the sequential, simultaneous, concurrent or coextensive administration of the disclosed antibodies and one or more chemotherapeutic agents. Particularly preferred embodiments of this aspect of the invention will comprise the administration of a radiolabeled antibody.

While the antibodies of the invention may be administered as described immediately above, it must be emphasized that in other embodiments conjugated and unconjugated antibodies may be administered to otherwise healthy patients as a first line therapeutic agent. In such embodiments the antibodies may be administered to patients having normal or average red marrow reserves and/or to patients that have not, and are not, undergoing adjunct therapies such as external beam radiation or chemotherapy.

However, as discussed above, selected embodiments of the invention comprise the administration of antibodies to myelosuppressed patients or in combination or conjunction with one or more adjunct therapies such as radiotherapy or chemotherapy (i.e. a combined therapeutic regimen). As used herein, the administration of antibodies in conjunction or combination with an adjunct therapy means the sequential, simultaneous, coextensive, concurrent, concomitant or contemporaneous administration or application of the therapy and the disclosed antibodies. Those skilled in the art will appreciate that the administration or application of the various components of the combined therapeutic regimen may be timed to enhance the overall effectiveness of the treatment. For example, chemotherapeutic agents could be administered in standard, well known courses of treatment followed within a few weeks by radioimmunoconjugates of the present invention. Conversely, cytotoxin associated antibodies could be administered intravenously followed by tumor localized external beam radiation. In yet other embodiments, the antibody may be administered concurrently with one or more selected chemotherapeutic agents in a single office visit. A skilled artisan (e.g. an experienced oncologist) would be readily be able to discern effective combined therapeutic regimens without undue experimentation based on the selected adjunct therapy and the teachings of the instant specification.

In this regard it will be appreciated that the combination of the antibody (with or without cytotoxin) and the chemotherapeutic agent may be administered in any order and within any time frame that provides a therapeutic benefit to the patient. That is, the chemotherapeutic agent and antibody may be administered in any order or concurrently. In selected embodiments the antibodies of the present invention will be administered to patients that have previously undergone chemotherapy. In yet other embodiments, the antibodies and the chemotherapeutic treatment will be administered substantially simultaneously or concurrently. For example, the patient may be given the modified antibody while undergoing a course of chemotherapy. In preferred embodiments the modified antibody will be administered within 1 year of any chemotherapeutic agent or treatment. In other preferred embodiments the antibody will be administered within 10, 8, 6, 4, or 2 months of any chemotherapeutic agent or treatment. In still other preferred embodiments the antibody will be administered within 4, 3, 2 or 1 week of any chemotherapeutic agent or treatment. In yet other embodiments the antibody will be administered within 5, 4, 3, 2 or 1 days of the selected chemotherapeutic agent or treatment. It will further be appreciated that the two agents or treatments may be administered to the patient within a matter of hours or minutes (i.e. substantially simultaneously).

Moreover, in accordance with the present invention a myelosuppressed patient shall be held to mean any patient exhibiting lowered blood counts. Those skilled in the art will appreciate that there are several blood count parameters conventionally used as clinical indicators of myelosuppresion and one can easily measure the extent to which myelosuppresion is occurring in a patient. Examples of art accepted myelosuppression measurements are the Absolute Neutrophil Count (ANC) or platelet count. Such myelosuppression or partial myeloablation may be a result of various biochemical disorders or diseases or, more likely, as the result of prior chemotherapy or radiotherapy. In this respect, those skilled in the art will appreciate that patients who have undergone traditional chemotherapy typically exhibit reduced red marrow reserves. As discussed above, such subjects often cannot be treated using optimal levels of cytotoxin (i.e. radionuclides) due to unacceptable side effects such as anemia or immunosuppression that result in increased mortality or morbidity.

More specifically conjugated or unconjugated antibodies of the present invention may be used to effectively treat patients having ANCs lower than about 2000/mm³ or platelet counts lower than about 150,000/mm³. More preferably the antibodies of the present invention may be used to treat patients having ANCs of less than about 1500/mm³, less than about 1000/mm³ or even more preferably less than about 500/mm³. Similarly, the antibodies of the present invention may be used to treat patients having a platelet count of less than about 75,000/mm³, less than about 50,000/mm³ or even less than about 10,000/mm³. In a more general sense, those skilled in the art will easily be able to determine when a patient is myelosuppressed using government implemented guidelines and procedures.

As indicated above, many myelosuppressed patients have undergone courses of treatment including chemotherapy, implant radiotherapy or external beam radiotherapy. In the case of the latter, an external radiation source is for local irradiation of a malignancy. For radiotherapy implantation methods, radioactive reagents are surgically located within the malignancy, thereby selectively irradiating the site of the disease. In any event, the disclosed antibodies may be used to treat disorders in patients exhibiting myelosuppression regardless of the cause.

In this regard it will further be appreciated that the antibodies of the instant invention may be used in conjunction or combination with any chemotherapeutic agent or agents (e.g. to provide a combined therapeutic regimen) that eliminates, reduces, inhibits or controls the growth of neoplastic cells in vivo. As discussed, such agents often result in the reduction of red marrow reserves. This reduction may be offset, in whole or in part, by the diminished myelotoxicity of the compounds of the present invention that advantageously allow for the aggressive treatment of neoplasms in such patients. In other preferred embodiments the radiolabeled immunoconjugates disclosed herein may be effectively used with radiosensitizers that increase the susceptibility of the neoplastic cells to radionuclides. For example, radiosensitizing compounds may be administered after the radiolabeled modified antibody has been largely cleared from the bloodstream but still remains at therapeutically effective levels at the site of the tumor or tumors.

With respect to these aspects of the invention, exemplary chemotherapic agents that are compatible with the instant invention include alkylating agents, vinca alkaloids (e.g., vincristine and vinblastine), procarbazine, methotrexate and prednisone. The four-drug combination MOPP (mechlethamine (nitrogen mustard), vincristine (Oncovin), procarbazine and prednisone) is very effective in treating various types of lymphoma and comprises a preferred embodiment of the present invention. In MOPP-resistant patients, ABVD (e.g., adriamycin, bleomycin, vinblastine and dacarbazine), ChIVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphomas, in HARRISON'S PRINCIPLES OF INTERNAL MEDICINE 1774-1788 (Kurt J. Isselbacher et al., eds., 13^{th} ed. 1994) and V. T. DeVita et al., (1997) and the references cited therein for standard dosing and scheduling. These therapies can be used unchanged, or altered as needed for a particular patient, in combination with one or more modified antibodies as described herein.

Additional regimens that are useful in the context of the present invention include use of single alkylating agents such as cyclophosphamide or chlorambucil, or combinations such as CVP (cyclophosphamide, vincristine and prednisone), CHOP (CVP and doxorubicin), C-MOPP (cyclophosphamide, vincristine, prednisone and procarbazine), CAP-BOP (CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). Those skilled in the art will readily be able to determine standard dosages and scheduling for each of these regimens. CHOP has also been combined with bleomycin, methotrexate, procarbazine, nitrogen mustard, cytosine arabinoside and etoposide. Other compatible chemotherapeutic agents include, but are not limited to, 2-chlorodeoxyadenosine (2-CDA), 2′-deoxycoformycin and fludarabine.

For patients with intermediate- and high-grade NHL, who fail to achieve remission or relapse, salvage therapy is used. Salvage therapies employ drugs such as cytosine arabinoside, cisplatin, etoposide and ifosfamide given alone or in combination. In relapsed or aggressive forms of certain neoplastic disorders the following protocols are often used: IMVP-16 (ifosfamide, methotrexate and etoposide), MIME (methyl-gag, ifosfamide, methotrexate and etoposide), DHAP (dexamethasone, high dose cytarabine and cisplatin), ESHAP (etoposide, methylpredisolone, HD cytarabine, cisplatin), CEPP(B) (cyclophosphamide, etoposide, procarbazine, prednisone and bleomycin) and CAMP (lomustine, mitoxantrone, cytarabine and prednisone) each with well known dosing rates and schedules.

The amount of chemotherapeutic agent to be used in combination with the antibodies of the instant invention may vary by subject or may be administered according to what is known in the art. See for example, Bruce A Chabner et al., Antineoplastic Agents, in GOODMAN & GILMAN'S THE PHARMACOLOGICAL BASIS OF THERAPEUTICS 1233-1287 ((Joel G. Hardman et al., eds., 9^{th} ed. 1996).

As previously discussed, the antibodies of the present invention, immunoreactive fragments or recombinants thereof may be administered in a pharmaceutically effective amount for the in vivo treatment of mammalian disorders. In this regard, it will be appreciated that the disclosed antibodies will be formulated so as to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of the antibody, immunoreactive fragment or recombinant thereof, conjugated or unconjugated to a therapeutic agent, shall be held to mean an amount sufficient to achieve effective binding with selected immunoreactive antigens on neoplastic or immunoreactive cells and provide for an increase in the death of those cells. Of course, the pharmaceutical compositions of the present invention may be administered in single or multiple doses to provide for a pharmaceutically effective amount of the antibody.

More specifically, they the disclosed antibodies and methods should be useful for reducing tumor size, inhibiting tumor growth and/or prolonging the survival time of tumor-bearing animals. Accordingly, this invention also relates to a method of treating tumors in a human or other animal by administering to such human or animal an effective, non-toxic amount of antibody. One skilled in the art would be able, by routine experimentation, to determine what an effective, non-toxic amount of antibody would be for the purpose of treating malignancies. For example, a therapeutically active amount of a antibody may vary according to factors such as the disease stage (e.g., stage I versus stage IV), age, sex, medical complications (e.g., immunosuppressed conditions or diseases) and weight of the subject, and the ability of the antibody to elicit a desired response in the subject. The dosage regimen may be adjusted to provide the optimum therapeutic response. For example, several divided doses may be administered daily, or the dose may be proportionally reduced as indicated by the exigencies of the therapeutic situation. Generally, however, an effective dosage is expected to be in the range of about 0.05 to 100 milligrams per kilogram body weight per day and more preferably from about 0.5 to 10, milligrams per kilogram body weight per day.

For purposes of clarification “Mammal” refers to any animal classified as a mammal, including humans, domestic and farm animals, and zoo, sports, or pet animals, such as dogs, horses, cats, cows, etc. Preferably, the mammal is human.

“Treatment” refers to both therapeutic treatment and prophylactic or preventative measures. Those in need of treatment include those already with the disease or disorder as well as those in which the disease or disorder is to be prevented. Hence, the mammal may have been diagnosed as having the disease or disorder or may be predisposed or susceptible to the disease.

In keeping with the scope of the present disclosure, the antibodies of the invention may be administered to a human or other animal in accordance with the aforementioned methods of treatment in an amount sufficient to produce such effect to a therapeutic or prophylactic degree. The antibodies of the invention can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of antibodies according to the present invention may prove to be particularly effective.

Methods of preparing and administering conjugates of the antibody, immunoreactive fragments or recombinants thereof, and a therapeutic agent are well known to or readily determined by those skilled in the art. The route of administration of the antibody (or fragment thereof) of the invention may be oral, parenteral, by inhalation or topical. The term parenteral as used herein includes intravenous, intraarterial, intraperitoneal, intramuscular, subcutaneous, rectal or vaginal administration. The intravenous, intraarterial, subcutaneous and intramuscular forms of parenteral administration are generally preferred. While all these forms of administration are clearly contemplated as being within the scope of the invention, a preferred administration form would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumine), etc. However, in other methods compatible with the teachings herein, the antibodies can be delivered directly to the site of the adverse cellular population thereby increasing the exposure of the diseased tissue to the therapeutic agent.

Preparations for parenteral administration includes sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a antibody by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 and U.S. Ser. No. 09/259,338 each of which is incorporated herein by reference. Such articles of manufacture will preferably have labels or package inserts indicating that the associated compositions are useful for treating a subject suffering from, or predisposed to autoimmune or neoplastic disorders.

As discussed in detail above, preferred embodiments of the present invention provide compounds, compositions, kits and methods for the treatment of neoplastic disorders in a mammalian subject in need of treatment thereof. Preferably, the subject is a human. The neoplastic disorder (e.g., cancers and malignancies) may comprise solid tumors such as melanomas, gliomas, sarcomas, and carcinomas as well as myeloid or hematologic malignancies such as lymphomas and leukemias. In general, the disclosed invention may be used to prophylactically or therapeutically treat any neoplasm comprising an antigenic marker that allows for the targeting of the cancerous cells by the modified antibody. Exemplary cancers that may be treated include, but are not limited to, prostate, gastric carcinomas such as colon, skin, breast, ovarian, lung and pancreatic More particularly, the antibodies of the instant invention may be used to treat Kaposi's sarcoma, CNS neoplasms (capillary hemangioblastomas, meningiomas and cerebral metastases), melanoma, gastrointestinal and renal sarcomas, rhabdomyosarcoma, glioblastoma (preferably glioblastoma multiforme), leiomyosarcoma, retinoblastoma, papillary cystadenocarcinoma of the ovary, Wilm's tumor or small cell lung carcinoma. It will be appreciated that appropriate antibodies may be derived for tumor associated antigens related to each of the forgoing neoplasms without undue experimentation in view of the instant disclosure.

Exemplary hematologic malignancies that are amenable to treatment with the disclosed invention include Hodgkins and non-Hodgkins lymphoma as well as leukemias, including ALL-L3 (Burkift's type leukemia), chronic lymphocytic leukemia (CLL) and monocytic cell leukemias. It will be appreciated that the compounds and methods of the present invention are particularly effective in treating a variety of B-cell lymphomas, including low grade/follicular non-Hodgkin's lymphoma (NHL), cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL and Waldenstrom's Macroglobulinemia. It should be clear to those of skill in the art that these lymphomas will often have different names due to changing systems of classification, and that patients having lymphomas classified under different names may also benefit from the combined therapeutic regimens of the present invention. In addition to the aforementioned neoplastic disorders, it will be appreciated that the disclosed invention may advantageously be used to treat additional malignancies bearing compatible tumor associated antigens.

Besides neoplastic disorders, the antibodies of the instant invention are particularly effective in the treatment of autoimmune disorders or abnormal immune responses. In this regard, it will be appreciated that the antibodies may be used to control, suppress, modulate or eliminate unwanted immune responses to both external and autoantigens. For example, in one embodiment, the antigen is an autoantigen. In another embodiment, the antigen is an allergan. In yet other embodiments, the antigen is an alloantigen or xenoantigen. Use of the disclosed antibodies to reduce an immune response to alloantigens and xenoantigens is of particular use in transplantation, for example to inhibit rejection by a transplant recipient of a donor graft, e.g. a tissue or organ graft or bone marrow transplant. Additionally, suppression or elimination of donor T cells within a bone marrow graft is useful for inhibiting graft versus host disease.

In yet other embodiments the antibodies of the present invention may be used to treat immune disorders that include, but are not limited to, allergic bronchopulmonary aspergillosis; Allergic rhinitis Autoimmune hemolytic anemia; Acanthosis nigricans; Allergic contact dermatitis; Addison's disease; Atopic dermatitis; Alopecia areata; Alopecia universalis; Amyloidosis; Anaphylactoid purpura; Anaphylactoid reaction; Aplastic anemia; Angioedema, hereditary; Angioedema, idiopathic; Ankylosing spondylitis; Arteritis, cranial; Arteritis, giant cell; Arteritis, Takayasu's; Arteritis, temporal; Asthma; Ataxia-telangiectasia; Autoimmune oophoritis; Autoimmune orchitis; Autoimmune polyendocrine failure; Behcet's disease; Berger's disease; Buerger's disease; bronchitis; Bullous pemphigus; Candidiasis, chronic mucocutaneous; Caplan's syndrome; Post-myocardial infarction syndrome; Post-pericardiotomy syndrome; Carditis; Celiac sprue; Chagas's disease; Chediak-Higashi syndrome; Churg-Strauss disease; Cogan's syndrome; Cold agglutinin disease; CREST syndrome; Crohn's disease; Cryoglobulinemia; Cryptogenic fibrosing alveolitis; Dermatitis herpetifomis; Dermatomyositis; Diabetes mellitus; Diamond-Blackfan syndrome; DiGeorge syndrome; Discoid lupus erythematosus; Eosinophilic fasciitis; Episcleritis; Drythema elevatum diutinum; Erythema marginatum; Erythema multiforme; Erythema nodosum; Familial Mediterranean fever; Felty's syndrome; Fibrosis pulmonary; Glomerulonephritis, anaphylactoid; Glomerulonephritis, autoimmune; Glomerulonephritis, post-streptococcal; Glomerulonephritis, post-transplantation; Glomerulopathy, membranous; Goodpasture's syndrome; Granulocytopenia, immune-mediated; Granuloma annulare; Granulomatosis, allergic; Granulomatous myositis; Grave's disease; Hashimoto's thyroiditis; Hemolytic disease of the newborn; Hemochromatosis, idiopathic; Henoch-Schoenlein purpura; Hepatitis, chronic active and chronic progressive; Histiocytosis X; Hypereosinophilic syndrome; Idiopathic thrombocytopenic purpura; Job's syndrome; Juvenile dermatomyositis; Juvenile rheumatoid arthritis (Juvenile chronic arthritis); Kawasaki's disease; Keratitis; Keratoconjunctivitis sicca; Landry-Guillain-Barre-Strohl syndrome; Leprosy, lepromatous; Loeffler's syndrome; lupus; Lyell's syndrome; Lyme disease; Lymphomatoid granulomatosis; Mastocytosis, systemic; Mixed connective tissue disease; Mononeuritis multiplex; Muckle-Wells syndrome; Mucocutaneous lymph node syndrome; Mucocutaneous lymph node syndrome; Multicentric reticulohistiocytosis; Multiple sclerosis; Myasthenia gravis; Mycosis fungoides; Necrotizing vasculitis, systemic; Nephrotic syndrome; Overlap syndrome; Panniculitis; Paroxysmal cold hemoglobinuria; Paroxysmal nocturnal hemoglobinuria; Pemphigoid; Pemphigus; Pemphigus erythematosus; Pemphigus foliaceus; Pemphigus vulgaris; Pigeon breeder's disease; Pneumonitis, hypersensitivity; Polyarteritis nodosa; Polymyalgia rheumatic; Polymyositis; Polyneuritis, idiopathic; Portuguese familial polyneuropathies; Pre-eclampsia/eclampsia; Primary biliary cirrhosis; Progressive systemic sclerosis (Scleroderma); Psoriasis; Psoriatic arthritis; Pulmonary alveolar proteinosis; Pulmonary fibrosis, Raynaud's phenomenon/syndrome; Reidel's thyroiditis; Reiter's syndrome, Relapsing polychrondritis; Rheumatic fever; Rheumatoid arthritis; Sarcoidosis; Scleritis; Sclerosing cholangitis; Serum sickness; Sezary syndrome; Sjogren's syndrome; Stevens-Johnson syndrome; Still's disease; Subacute sclerosing panencephalitis; Sympathetic ophthalmia; Systemic lupus erythematosus; Transplant rejection; Ulcerative colitis; Undifferentiated connective tissue disease; Urticaria, chronic; Urticaria, cold; Uveitis; Vitiligo; Weber-Christian disease; Wegener's granulomatosis and Wiskoft-Aldrich syndrome.

The foregoing description will be more fully understood with reference to the following examples. Such examples, are, however, demonstrative of preferred methods of practicing the present invention and are not limiting of the scope of the invention or the claims appended hereto.

### EXAMPLE

### MATERIALS AND METHODS

### Cell Lines

A recombinant CHO production cell (50C9) expressing the anti-CD20 chimeric antibody Rituxan® was used in co-expression studies with rat GnTIII. A CHO cell line constructed in our laboratory expressing membrane bound FcγRI and a mouse fibroblast cell line expressing FcγRIIa (ref) were used in whole cell binding studies. Sorted NK cells were obtained from AllCells Inc. SKW6.4 (Ralph et al. 1983) cells, a cell line expressing CD20 was used as a target in ADCC activity assays.

### Establishment of GnTIII/Anti-CD20 Cell Lines

An anti-CD20 production cell line (50C9) was electroporated with the vector pCIPGnT3 harboring the rat GnTIII gene a under a constitutive CMV promoter and a puromycin resistance gene (FIG. 1). Approximately 0.2-1 μg of DNA was electroporated with 4×10⁶ cells using a Biorad elecroporation device. The plasmid was previously digested with PvuI and Bst1107I (New England Biolabs) which separates the genes expressed in CHO cells from the portion used to propagate the plasmid in bacteria. Conditions for the electroporation were 210V, 400 μF, 13ω. Each electroporation was plated into 96 well plates and fed with CHO SfMII media (Gibco BRL), 50 nM MTX and 5 μg/mL puromycin (Invitrogen) after 2 days and every 2-3 days until puromycin resistant colonies arose. Resistant colonies were moved up to 6 well plates and then T25 flasks. GnTIII message RNA levels in puromycin resistant colonies were then quantitated using the relative QPCR kit (Ambion). Briefly, mRNA was prepared from 1×10⁷ cells using the RNEASY miniprep kit (Qiagen) and reverse transcribed with oligo dt and the cDNA Cycle® RT PCR kit. cDNA was then used as a template in a relative QPCR with GnTIII specific primers and 18s RNA primers were used to amplify the internal control.

### IgG Expression Levels

Expression levels from a three day run in 100 mL spinner flasks were calculated using an enzyme linked immunoadsorbant assay. IgG from cell line supernatants was captured with anti-human IgG (Roche) coated onto microtitre plates and detected using anti-human IgG (Fab′)₂ horse radish peroxidase conjugate (Roche). A standard curve was produced using dilutions of purified monoclonal IgGI. Expression levels were then converted to picogram per cell per day (pcd) based upon ELISA results and measured viable cell densities.

### Glycosylation Analysis

Antibody samples (300 μg) were buffer exchanged into 20 mM sodium phosphate containing 50 mM EDTA and 0.02% (w/v) sodium azide, pH 7.5 using a Microcon-30 concentrator. Five units of recombinant Peptide N-glycosidase F (Glyko) were added to the samples and incubated for approximately 15 hours at 37° C. Following the digestion, 50 uL of 20 mM sodium phosphate, 50 mM ED TA, 0.02% (w/v) sodium azide, pH 7.5 was added to each sample. The de-glycosylated proteins were precipitated by heating at 95° C. for 5 minutes and removed by centrifugation at 9,000×g for 10 minutes. The supernatant containing the released oligosaccharides were dried in a centrifugal vacuum evaporator and labeled by the addition of 15 μL of 10 mg/mL 9-aminopyrene-1,4,6-trisulfonate (APTS, Beckman) in 15% acetic acid and 5 μL of 1 M sodium cyanoborohydride in tetrahydrofuran. The labeling reaction was incubated at 55° C. for approximately 2 hours then diluted with 500 gL of water. Samples were diluted 1:4 with acetonitrile prior to HPLC analysis.

The PNGAse released N-linked oligosaccharides were analyzed by NP-HPLC. The method used a TosoHaas Amide-80 column (4.6×250 mM, 5 μm particle size, 900 Å pore size) on a Beckman 126 HPLC system with Gold Nouveau software and a Jasco FP-920 fluorescence detector. The eluant system consisted of 0.1% acetic acid in acetonitrile (Buffer A); 0.2% acetic acid, 0.2% triethylamine in water (Buffer B), at a flow rate of 1.0 mL/min. The elution profile was monitored by fluorescence detection with excitation at 488 nm and emission at 520 nm. The column was equilibrated with 28% Buffer B and a sample (50 μL) of IgG N-linked oligosaccharides was injected and held for 15 minutes at 28% Buffer B. The N-linked oligosaccharides were resolved with a linear gradient from 28% Buffer B to 38% Buffer B over 50 minutes, to 90% over 1 minute and held for 9 minutes, to 28% Buffer B in 1 minute and held for 14 minutes for equilibration prior to the next injection.

### ADCC Activity Assay

Peripheral blood mononuclear cells (PBMC) were separated from either heparinized fresh human blood (or buffy coat) by standard centrifugation procedures on FicoII/Hypaque (Sigma). The PBMC used as effector cells, were activated by culturing in 10% FBS in RPMI (Gibco. BRL) with 10 U/mL of IL-2 (Roche) for overnight. SKW 6.4 cells were grown in log phase and re-suspended at 4×10⁵/mL after washed in assay medium (2% FBS in Phenol red-free RPMI medium). The target cells (SKW6.4) were added to the plate at 50 μL/well into 96-well Flat-bottomed tissue culture plate. Antibodies were serially diluted with assay medium, then added 50 μL/well to triplicate wells in the plate. The plates were incubated at room temperature for 10 minutes prior to the addition of 100 μL of PBMC effector cells at a concentration of 16×10⁶/mL. The cell mixtures with antibodies were incubated at 37° C. for 4 hours in a humid COZ incubator. The supernatant of 100 μL was removed from each well and analyzed by measuring LDH activity released from damaged target cells (LDH Cytotoxicity Detection Kit, Roche Molecular biochemicals, Germany). The effector cells or/and target cells alone were also included as controls. The specific lysis was calculated relative to a total lysis control, resulting from incubating the target cells with 100 μL of 2% Triton X-100.

For the ADCC blocking experiments the procedure was the same as outlined above except anti-FcγRIII (clone LNK16, Serotec) or anti-FcγRI (clone 10.1 Serotec) at concentrations of 5 μg/mL were pre-incubated with the PBMC effector cells for 20 minutes and washed twice with assay medium before use in the assay.

### Whole Cell ELISA

FcγRI expressing CHO cells, a mouse fibroblast cell line expressing FcγRIIa and sorted FcγRIII positive NK cells were covalently attached to a microtitre plate at 1×10⁵ cells per well using gluteraldehyde as a cross-linker. Antibody dilutions were added to the plates in triplicate in blocking buffer (Phosphate Buffered Saline, 0.5% dry milk powder and 0.01% Thimersol) and left to bind at 37° C. for 1 hour. Following a several washes with PBS the bound antibody was detected using anti-human IgG Fab fragments conjugated with horse radish peroxidase (Roche) and visualized after addition of tetramethylbenzidine (Sigma). Both antibodies are reported to block IgG Fc binding to the relevant receptor

We constructed a constitutive expression plasmid for GnTIII. The plasmid pCIPGnT3 (FIG. 1) contains the rat GnTIII gene under a constitutive CMV promoter and bovine growth hormone polyadenylation region. The plasmid also contains a puromycin resitance gene, which allows selection of in puromycin containing media. Following electroporation of the Rituxan® producing cell line (50C9) with the plasmid, puromycin resistant colonies were obtained. We then employed a relative QPCR assay to detect message RNA levels in the resistant colonies to decide which clones to study further. Most of the clones isolated expressed high levels of GnTIII message and a few clones expressed at much lower levels. An example of a relative QPCR experiment is shown in FIG. 2. Clone 50C9-IA7 is an example of a clone which expresses at a lower level whilst 50C9-1A12 and 50C9-1B9 are clones which express at much higher levels. No GnTIII message was detected for the parent cell line (not transfected with the pCIPGnT3 plasmid) indicating the absence of endogenous GnTIII expression in this cell line.

Three clones were then chosen to study the in-vivo catalytic effects of GnTIII on the glycoforms of purified antibody by HPLC analysis. All three showed considerable glycoform variation when compare to the parent (50C9) cell line. A typical HPLC trace for the parent and one GnTIII transfectant clone is shown in FIG. 3. No bisected glycoforms were found for the 50C9 cell line. However, for the GnTIII positive cell line the majority of glycoforms (48-71%) contain a bisected GlcNac residue. A full set of results for the three cell lines are shown in Table 1. The data shows that there are small differences in the glycoform composition of GnTIII transfected clones but that for all three the dominant glycoform species found was a bisected biantennary oligosaccharide with one galactose residue (G1+G1cNAc). Only small amounts (3-5%) of bisected biantennary oligosaccharide with two galactose residues were detected (G2+G1cNAc).

We also studied the growth kinetics and production levels in the three GnTIII positive cell lines and compared them with 50C9 which produces large amounts of immunolglobulin (pcd) and has very good growth kinetics (td). We found all three cell lines to have very good expression levels of immunolglobulin. Furthermore, all three cell lines also have favorable growth kinetics and one clone (50C9-1A12) has growth kinetics (i.e. doubling time) very similar to the parent antibody (Table II). The summary of data shown in Table II indicates no correlation between mRNA levels and GnTIII activity or doubling time in the cell lines.

The high mRNA levels and GnTIII activity found in clone 50C9-1A12 do not seem to effect the growth kinetics or antibody expression levels. These results differ from those published previously in which the level of GnTIII expression correlated to growth inhibition. (Umana et. al. 1999b). Two possible reasons are suggested for the growth inhibition effect. Either a direct effect of protein over-expression leading to inhibition which is independent of the catalytic activity of GnTIII or a direct effect of the in-vivo catalytic activity of GnTIII on endogenous proteins. The previous work in this field has used glycotransferase co-expression in cell lines expressing only small amounts of recombinant protein.

### Increased ADCC Correlates with Increased Binding to FcγRIII

The anti-CD20 antibody used in this study is approved as a therapeutic agent in non-hodgkins lymphoma and has been shown to produce effective responses in approximately 50% of patients through depletion of normal and malignant B cells. The possible mechanisms include complement dependant cytotoxicity (CDC), antibody dependant cellular cytotoxicity (ADCC) and induction of apoptosis of CD20 positive cells on binding of the antibody. The over-expression of GnTIII has led to the isolation of clones with bisecting glycoform hybrids which are absent in the parent. These bisecting glycoforms have been implicated in the biological activity of some antibodies and so the antibodies purified from the 50C9/GnTIII clones were studied for changes in biological activity. No differences due to complement binding or apoptosis of CD20 positive cells on antibody binding were observed for the glycoform altered antibodies (data not shown). However, antibodies produced by all three GnTIII transfected cell lines studied were as effective as antibodies produced by the wild type cell line in killing CD20 positive target cells but at a 10 to 20 times lower concentration. This agrees with results reported for the over-expression of GnTIII in a cell line expressing an anti-neuroblastoma IgG.(Umana et. al. 1999a) In the aforementioned report the glycoforms of an antibody with low ADCC activity were altered resulting in higher ADCC activity and making it more attractive for therapeutic use.(Umana et. al. 1999a)

Here we have taken an approved therapeutic antibody with good ADCC activity and improved it further. This may allow the use of the antibody at lower doses with no reduction in efficacy. Moreover, the higher ADCC activity at a lower antibody concentration may result in an enhanced response in lymphomas and leukemias expressing lower levels of the CD20 antigen. Certain forms of these diseases require high doses of the current drug to be effective. The work also suggests that other antibodies deficient in the bisecting glycoforms may improve their in vivo cytolytic function.

We used an anti-FcγRIII antibody (reported to block Fc binding) to specifically block the FcγRIII receptors on NK cells and study the effect on ADCC activity. Blocking NK cells with the anti-FcγRIII antibody abolished the ADCC activity of both 50C9 and 50C9-1A7. FIG. 5 shows the results obtained from the 50C9-1A7 antibody preparation. No inhibition of ADCC was observed in an experiment in which PBMC cells were pre-incubated with an antibody against FcγRI which is reported to block Fc binding to the FcγRI receptor. The data suggests therefore that FcγRI receptors are not involved in the ADCC activity of the 50C9-IA7 antibody preparation with bisected biantennary oligosaccarides.

Using a whole cell ELISA, the binding characteristics of the parent and the GnTIII positive clone 50C9-1A7 were evaluated. Antibodies from 50C9-1A7 bound better to NK cells than antibodies prepared from the parent 50C9 (FIG. 6). The antibody also bound better to FcγRI and FcγRII expressing cells (results not shown). However, the role of FcγRI in ADCC has been discounted in the previously described experiment (FIG. 5). The increase in ADCC activity is therefore most likely due to increased binding of the antibody to FcyRIII on NK cells. Since no CD20 antigen was present in the ELISA, increased binding due to better crosslinking of IgG can be ruled out. Presumably the increase in binding is due to conformational effects specified by the bisecting glycoform on the Fc structure of the antibody. Since the parent antibody already has good ADCC activity it probably has a near optimal confirmation for FcyRIII binding which is then ‘fine tuned’ by the addition of the bisecting G1cNAc in the N-linked bianntennary oligosaccharide structure.

Table I. Results of glycoform analysis by HPLC. Samples were run as described in the text. The percentages of each species was calculated by peak areas. G0=Non bisected biantennary complex with no galactose residues. G0+G1cNAc=Bisected biatennary complex with no Galactose G1=Non bisected biantennary complex with 1 galactose residue. G1+GlcNAc=Bisected biantennary complex with 1 Galactose residue. G2=Non bisected biantennary complex with 2 galactose residues. G2+G1cNAc=Bisected biantennary complex with 2 Galactose residues. Structures are shown in FIG. 3.

TABLE I
Percentage of antibody preparations with Bisected glycoforms

| | | G0 + | | | | |
|---|---|---|---|---|---|---|
| Clone | G0 | GlcNAc | G1 | G1 + GlcNAc | G2 | G2 + GlcNAc |
| 50C9-1A12 | 8 | 15 | 15 | 30 | 4 | 3 |
| 50C9-1A7 | 5 | 17 | 11 | 35 | 4 | 3 |
| 50C9-1B9 | 1 | 20 | 6 | 46 | 3 | 5 |
| 50C9 | 42 | 0 | 47 | 0 | 11 | 0 |

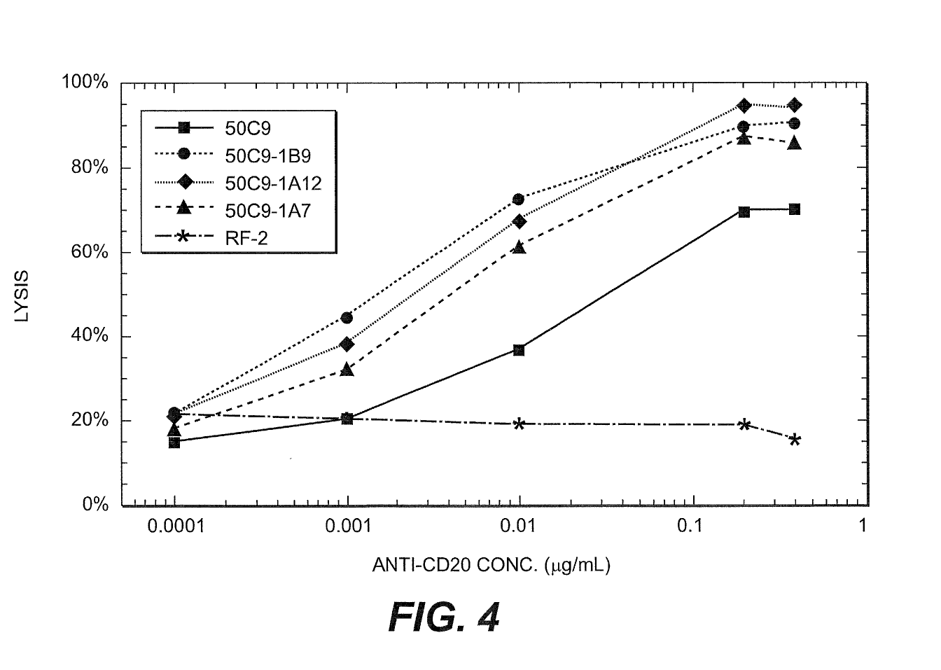

Table II. Summery of GnTIII clones. Td=Double time of clones in hours; Pcd=(picogrm per cell per day. Antibody expression levels measured by ELISA; % GlcNAc=percentage of antibody oligosaccharide with bisected G1cNAc's determined by the addition of GO+G1cNAc, G1+G1cNAc and G2+G1cNAc from table 1. GnTIII mRNA=mRNA levels as measured by relative QPCR (FIG. 2); [Ab] for 50% lysis=Antibody concentration for 50% lysis of target cells in an ADCC assay. Measured from extrapolation from FIG. 4 (in ng/mL)

TABLE II
Summary of 50C9-GnTlll clones

| | | | | | [Ab] for 50% |
|---|---|---|---|---|---|
| | | | | GnTlll | lysis |
| Clone | Td (hrs) | Pcd | % GlcNAc | mRNA | (ng.ml⁻¹) |
| 50C9-1A12 | 27 | 72 | 48 | High | 3 |
| 50C9-1A7 | 45 | 90 | 55 | low | 1.5 |
| 50C9-1B9 | 40 | 102 | 71 | High | 4 |
| 50C9 | 24 | 75 | 0 | None | 35 |

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

Further illustrations of the invention are described in “Expression of GnTIII in a recombinant anti-CD20 CHO production cell line: Expression of antibodies with altered glycoforms leads to an increase in ADCC through higher affinity for FC gamma RIII,” Davies J, Jiang L, Pan L Z, LaBarre M J, Anderson D, Reff M.; Biotechnol Bioeng, Aug. 20, 2001; 74(4):288-94, the contents of which are incorporated herein by reference in their entirety.

### REFERENCES

Bailey J E, Umana P, Minch S, et al. 1997. Metabolic engineering of n-linked glycoform synthesis systems in Chinese hamster ovary (CHO) cells. Animal Cell Technology 489-494.

Gavilondo J V, Larrick J W. 2000. Antibody Engineering at the Millenium. Biotechniques 29:128-145.

Hollinger P, Bohlen H. 1999. Engineering antibodies for the clinic. Cancer and Metastatis Reviews 18:411-419.

Lifely M R, Hale C, Boyce S, et al. 1995. Glycosylation and biological activity of CAMPATH-1H expressed in different cell lines and grown under different culture conditions. Glycobiology 5:813-822.

Maloney D G, Grillo-Lopez A J, White C A, et al. 1997. IDEC-C2138 (Rituximab) Anti-CD20 Monoclonal Antibody Therapy in Patients with Relapsed Low-Grade Non-Hodgkin's Lymphoma. Blood 90:2188-2195.

Narisimhan S. 1982. Control of glycoprotein synthesis. UDP-G1cNAc:glycopeptide bet 4-Nacetylglucosaminyltransferase III, an enzyme in hen oviduct which adds G1cNAc in beta 1-4 linkage to the beta-linked mannose of the trimannosyl core of N-glycosyl oligosaccharides. J Biol Chem 257:10235-10242.

Newman R, Ryskamp T. The Evolution of MAbs from Research Reagents to Mainstream Commercial Therapeutics. In: Maurizio Zanetti and Donald J Capra editor. The Antibodies. Amsterdam, The Netherlands: Harwood Academic Publishers. p. 1-42.

Ralph P, Saiki O, Maurer D H, et al. 1983. IgM and IgG Secretion in Human B-Cell Lines Regulated by B-Cell-Inducing Factors (BIF) and Phorbol Ester. Immunol Lett 7:17-23.

Reff M E, Carner K, Chambers K S, et al. 1994. Depletion of B cells in vivo by a chimeric mouse human monoclonal antibody to CD20. Blood 83:435-445.

Shan D, Ledbetter J A, Press O W. 1998. Apoptosis of Malignant Human B Cells by Ligation of CD20 with Monoclonal Antibodies. Blood 91:1644-1652.

Umana P, Jean-Mairet J, Moudry R, et al. 1999a. Engineered glycoforms of an antineuroblastoma IgG 1 with optimized antibody-dependent cellular cytotoxic activity. Nature Biotechnology 17:176-180.

Umana P, Keam-Mairet J, Bailey J. 1999b. Tetracycline-regulated overexpression of glycosyltransferases in Chinese hamster ovary cells. Biotechnol Bioeng 65:542-549.

Valentine M A, Meier K, Rossie S, et al. 1989. Phosphorylation of the CD20 phosphoprotein in resting B lymphocytes. J Biol Chem 264:11282-11287.

What is claimed is:

1. A eukaryotic cell line that expresses GnTIII and a recombinant antibody.

2. The eukaryotic cell line of claim 1 which is mammalian.

3. The mammalian cell line of claim 2 which is a CHO cell line.

4. The CHO cell line of claim 3 wherein the antibody is a human, chimeric or humanized anti-CD20 antibody.

5. The CHO cell line of claim 6 wherein said antibody is of the IgG1 or IgG3 isotype.

6. The CHO cell line of claim 7 wherein said antibody is RITUXAN®.

7. The eukaryotic cell line of claim 1 wherein said antibody reacts with a tumor associated antigen.

8. The eukaryotic cell line of claim 7 wherein said tumor associated antigen is selected from the group consisting of CD2, CD3, CD5, CD6, CD7, MAGE-1, MAGE-3, MUC-1, HPV 16, HPV E6, HPV E7, TAG-72, CEA, L6-Antigen, CD19, CD20, CD22, CD37, CD52, HLA-DR, EGF receptor and HER2 Receptor.

9. An antibody produced by a cell line according to any one of claims 1-6.

10. An anti-CD20 antibody produced by a cell line according to any one of claims 1-6.

11. A treatment comprising use of an anti-CD20 antibody which comprises administration of an anti-CD20 antibody produced by a cell line according to one of claims 1-6.

12. The treatment of claim 11 which is to treat a B cell lymphoma, malignancy or leukemia.

13. The treatment of claim 12 which is for non-Hodgkin's lymphoma or chronic lymphocytic leukemia.

14. The treatment of claim 11 which is for an autoimmune disease, transplantation or graft-vs-host disease.

15. The treatment of claim 14 which is for a B cell mediated autoimmune disease.

16. The treatment of claim 15 wherein said disease is ITP or lupus.

17. A pharmaceutical composition containing an antibody produced from a cell line according to one of claims 1-6.

18. A method of treating a disorder in a mammal in need thereof comprising administering a therapeutically effective amount of an antibody produced by a cell line according to claims 1 to said mammal.

19. The method of claim 18 wherein said antibody is a modified antibody.

20. The method of claim 19 wherein said modified antibody has at least a portion of one constant region domain omitted.

21. The method of claim 19 wherein said modified antibody comprises a domain deleted antibody.

22. The method of claim 21 wherein said domain deleted antibody lacks a C_{H}2 domain.

23. The method of claim 18 wherein said antibody reacts with a tumor associated antigen.

24. The method of claim 23 wherein said tumor associated antigen is selected from the group consisting of CD2, CD3, CD5, CD6, CD7, MAGE-1, MAGE-3, MUC-1, HPV 16, HPV E6, HPV E7, TAG-72, CEA, L6-Antigen, CD19, CD20, CD22, CD37, CD52, HLA-DR, EGF receptor and HER2 Receptor.

25. The method of claim 18 wherein said antibody is associated with a cytotoxic agent.

26. The method of claim 25 wherein said cytotoxic agent comprises a radioisotope.

27. The method of claim 26 wherein said radioisotope is selected from the group consisting of ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re, and ¹⁸⁸Re.

28. The method of claim 18 wherein said disorder is a neoplastic disorder.

29. The method of claim 28 wherein said neoplastic disorder is selected from the group consisting of relapsed Hodgkin's disease, resistant Hodgkin's disease high grade, low grade and intermediate grade non-Hodgkin's lymphomas, B cell chronic lymphocytic leukemia (B-CLL), lymhoplasmacytoid lymphoma (LPL), mantle cell lymphoma (MCL), follicular lymphoma (FL), diffuse large cell lymphoma (DLCL), Burkitt's lymphoma (BL), AIDS-related lymphomas, monocytic B cell lymphoma, angioimmunoblastic lymphoadenopathy, small lymphocytic; follicular, diffuse large cell; diffuse small cleaved cell; large cell immunoblastic lymphoblastoma; small, non-cleaved; Burkitt's and non-Burkitt's; follicular, predominantly large cell; follicular, predominantly small cleaved cell; and follicular, mixed small cleaved and large cell lymphomas.

30. The method of claim 18 further comprising the administration of a chemotherapeutic agent.

31. The method of claim 30 wherein said chemotherapeutic agent comprises Rituxan.

32. The method of claim 18 wherein said disorder is an immune disorder.

33. A kit useful for the treatment of a mammal suffering from or predisposed to a disorder comprising at least one container having a antibody produced by the cell lines of claim 1 deposited therein and a label or an insert indicating that said antibody may be used to treat said disorder.

34. The kit of claim 33 wherein said antibody reacts with a tumor associated antigen.

35. The kit of claim 34 wherein said tumor associated antigen is selected from the group consisting of CD2, CD3, CD5, CD6, CD7, MAGE-1, MAGE-3, MUC-1, HPV 16, HPV E6, HPV E7, TAG-72, CEA, L6-Antigen, CD19, CD20, CD22, CD37, CD52, HLA-DR, EGF receptor and HER2 Receptor.

36. The kit of claim 35 wherein said antibody has at least a portion of one constant region domain omitted.

37. The kit of claim 35 wherein said antibody comprises a domain deleted antibody.

38. The kit of claim 37 wherein said domain deleted antibody lacks the C_{H}2 domain.

39. An antibody produced by a cell line according to claims 1.

40. The antibody of claim 39 wherein said antibody reacts with an autoantigen.

41. The antibody of claim 39 wherein the antibody reacts with a tumor associated antigen.

42. The antibody of claim 40 wherein said tumor associated antigen is selected from the group consisting of CD2, CD3, CD5, CD6, CD7, MAGE-1, MAGE-3, MUC-1, HPV 16, HPV E6, HPV E7, TAG-72, CEA, L6-Antigen, CD19, CD20, CD22, CD37, CD52, HLA-DR, EGF receptor and HER2 Receptor.

43. The antibody of claim 39 wherein said antibody has at least a portion of one constant region domain omitted.

44. The antibody of claim 39 wherein said antibody comprises a domain deleted antibody.

45. The antibody of claim 43 wherein said domain deleted antibody lacks a C_{H}2 domain.

46. The antibody of claim 39 wherein said antibody is associated with a cytotoxic agent.

47. The antibody of claim 46 wherein said cytotoxic agent comprises a radioisotope.

48. The antibody of claim 47 wherein said radioisotope is selected from the group consisting of ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re, and ¹⁸⁸Re.

49. A method for forming antibodies comprising the steps of:
culturing prokaryotic or eukaryotic host cells comprising DNA sequences encoding GnT11 and a recombinant antibody antibody whereby the host cell expresses GnT11 and the recombinant antibody;
allowing the host cell to express GnT11 and the recombinant antibody; and
recovering said antibodies from the host cell culture.

50. The method of claim 49 wherein said antibody has at least a portion of one constant region domain omitted.

51. The method of claim 49 wherein said antibodies comprise domain deleted antibodies.

52. The method of claim 51 wherein said domain deleted antibodies lack the C_{H}2 domain.

53. The method of claim 49 wherein said antibodies react with a tumor associated antigen.

54. The method of claim 53 wherein said tumor associated antigen is selected from the group consisting of CD2, CD3, CD5, CD6, CD7, MAGE-1, MAGE-3, MUC-1, HPV 16, HPV E6, HPV E7, TAG-72, CEA, L6-Antigen, CD19, CD20, CD22, CD37, CD52, HLA-DR, EGF receptor and HER2 Receptor.

55. The method of claim 54 wherein said tumor associated antigen is TAG-72.

56. The method of claim 49 wherein said host cells comprise CHO cells.

57. A polycistronic vector for expressing GnTIII and functional antibodies in eukaryotic host cells which vector comprises a polycistronic transcription system comprising a DNA sequence encoding GnTIII and the following elements operably linked in the 5′ to 3′ orientation:
(i) a promoter operable in a eukaryotic cell;
(ii) a DNA sequence encoding an antibody light chain which optimally comprises at its 5′ terminus a signal peptide coding sequence operable in eukaryotic cells which DNA sequence does not comprise at its 3′ end a poly A sequence and comprising a start and a stop codon at the 5′ and 3′ terminus of said DNA sequence;
(iii) an internal ribosome entry site (IRES) obtained from a member selected from the group consisting of a cardiovirus, a herpes virus and a poliovirus; and
(iv) at least one DNA sequence comprising the following elements (a) a DNA sequence encoding an antibody heavy chain wherein said DNA optimally comprises at its 5′ terminus a signal peptide coding sequence operable in eukaryotic cells and wherein said DNA sequence comprises a poly A sequence at its 3′ terminus only if the DNA sequence is the 3′ most coding sequence in the polycistron, and further comprises a start and stop codon at the 5′ and 3′ termini of said DNA coding sequence;
wherein the DNA sequence encoding the antibody light chain is expressed at a ratio ranging between 10:1 and 1:1 with respect to the DNA sequence encoding the antibody heavy chain.

58. The polycistronic vector of claim 57, wherein the DNA sequences encoding antibody heavy and light chain constant regions are of primate origin.

59. The polycistronic vector of claim 58, wherein the antibody heavy and light chain constant regions are of human origin.

60. The polycistronic vector of claim 57, wherein the DNA sequences encoding antibody heavy and light chain variable regions are of primate origin.

61. The polycistronic vector of claim 60, wherein the heavy and light chain variable regions are of human origin.

62. The polycistronic vector of claim 57, wherein the DNA sequences encoding antibody heavy and light chain variable regions are of murine origin.

63. The polycistronic vector of claim 57, wherein the eukaryotic promoter is a mammalian promoter or viral promoter.

64. The polycistronic vector of claim 63, wherein the promoter is a CMV promoter.

65. The polycistronic vector of claim 57, wherein the IRES is obtained from a cardiovirus.

66. The polycistronic vector of claim 65, wherein the cardiovirus is human encephalomyocarditis virus.

67. The polycistronic vector of claim 57, wherein the functional antibodies expressed by the polycistronic vector specifically bind to a tumor antigen, an antigen expressed on a B cell or an antigen expressed on a T cell.

68. The polycistronic vector of claim 57, wherein the functional antibodies expressed by the polycistronic vector specifically bind to an antigen selected from the group consisting of CD2, CD3, CD5, CD6, CD7, MAGE-1, MAGE-3, MUC-1, HPV 16, HPV E6, HPV E7, TAG-72, CEA, L6-Antigen, CD19, CD20, CD22, CD37, CD52, HLA-DR, EGF receptor and HER2 Receptor.

69. The polycistronic vector of claim 68, wherein the antigen is CD20.

70. The polycistronic vector of claim 68, wherein the functional antibody is a human, humanized or chimeric antibody specific to CD20.

71. The polycistronic vector of claim 68, wherein the antibody is rituximab.

72. A mammalian cell comprising a polycistronic vector according to claims 57, wherein the mammalian cell secretes about 10-50 picograms functional antibody.

73. The mammalian cell of claim 72, wherein the mammalian cell is a Chinese Hamster Ovary cell.

74. The mammalian cell of claim 72, wherein the mammalian cell is a member selected from the group consisting of baby hamster kidney cells, fibroblast cells and myeloma cells.

75. The method of claim 68, wherein the functional antibodies are produced in batch fed cell cultures.
